(19) United States
(12) Patent Application Publication
(10) Pub. No.: US 2009/0234431 A1
(43) Pub. Date: Sep. 17, 2009

(54) ARTERIOVENOUS GRAFT BLOOD FLOW CONTROLLERS AND METHODS

(75) Inventors: Judah Weinberger, Teaneck, NJ (US); Nick Gately, Lambertville, NJ (US); Mark Gelfand, New York, NY (US)

Correspondence Address: SCHWEGMAN, LUNDBERG & WOESSNER/COLUMBIA UNIVERSITY, P.O. BOX 2938, MINNEAPOLIS, MN, 55402, US

(73) Assignee: The Trustees of Columbia University in the City of New York, New York, NY (US)

(21) Appl. No.: 12/389,986

(22) Filed: Feb. 20, 2009

Related U.S. Application Data
(63) Continuation of application No. PCT/US2007/017910, filed on Aug. 13, 2007.
(60) Provisional application No. 60/823,242, filed on Aug. 22, 2006.
(60) Provisional application No. 61/086,716, filed on Aug. 6, 2008.

(51) Int. Cl. A61F 2/06

(52) U.S. Cl. 623/1.13; 623/1.31; 623/1.15

(57) ABSTRACT

Blood flow restrictors are discussed. In some examples, a restrictor apparatus includes a converging entry portion, a diverging exit portion, and optionally a narrowed portion therebetween to restrict the flow of blood through an arteriovenous graft from a subject's artery to a vein. The structure of the restrictor apparatus decreases the pressure and volume of blood flow between the artery and vein to reduce or prevent hyperplasia or stenosis on the venous side, an increased load on the heart, or blood steal, among other things. The restrictor apparatus can be separate from, but couplable to, the arteriovenous graft. The restrictor apparatus can be integral with the arteriovenous graft. In some examples, the restrictor apparatus can be inserted into the arteriovenous graft in a compressed size and shape, and subsequently be allowed to expand to an uncompressed size and shape. Methods of forming and using the restrictor apparatus are also discussed.

### RELATED APPLICATIONS

This application is a nationalization under 35 U.S.C. §111(a) of International Application No. PCT/US2007/017910, filed Aug. 13, 2007 and published as WO 2008/024224 on Feb. 28, 2008, which claimed priority under 35 U.S.C. §119(e) to U.S. Provisional Ser. No. 60/823,242, filed Aug. 22, 2006. This application further claims priority under 35 U.S.C. § 119(e) to U.S. Provisional Ser. No. 61/086,716, filed Aug. 6, 2008. These applications and publication are incorporated herein by reference and made a part hereof.

### TECHNICAL FIELD

This patent document pertains generally to vascular access systems, apparatuses, and methods. More particularly, but not by way of limitation, this patent document pertains to arteriovenous graft blood flow controllers and methods.

### BACKGROUND

A number of medical procedures, such as hemodialysis, chemotherapy, transfusions, etc., require repeated access to a subject's vascular anatomy. In hemodialysis, for example, blood is removed from the subject's artery, treated with a dialysis machine that cleanses the blood of toxins (such as potassium and urea, as well as free water), and introduced back into the subject at a vein. Hemodialysis is typically conducted in a dedicated facility, either in a special room in a hospital or a clinic that specializes in hemodialysis. Hemodialysis sessions typically last about 3-6 hours and occur about 3 times per week for the duration of the subject's life or until the subject receives a kidney transplant.

For hemodialysis to be effective, large volumes of blood must be removed rapidly from the subject's body, passed through the dialysis machine, and returned to the subject. A number of operations have been developed to provide access to the circulatory system of a subject to connect the subject to the dialysis machine. The three primary modes of access to the blood in hemodialysis include an intravenous catheter, an arteriovenous fistula, or an arteriovenous graft. The type of access is typically influenced by factors such as the degree of the subject's renal (i.e., kidney) failure or the condition of his or her vasculature.

Catheter access typically consists of a plastic catheter with two lumens. The catheter is inserted into a large vein (typically in a limb) to allow withdrawal of relatively large flows of blood using one lumen. This blood is fed through the dialysis device, and returned to the subject via the other lumen. However, using the catheter access mode almost always allows less blood flow than that of a well functioning arteriovenous fistula or graft.

Arteriovenous fistulas and grafts comprise second and third modes, respectively, of access to blood in hemodialysis. To create an arteriovenous fistula, a vascular surgeon joins an artery and a vein together (typically in an upper extremity) through anastomosis. Since this bypasses the capillaries, blood flows at a very high rate through the arteriovenous fistula as compared to typical vessel flow. During treatment, two needles or cannulas are inserted into the arteriovenous fistula, one to draw blood and the other to return it. The advantages of arteriovenous fistula use include relative absence of a potential foreign body reaction, as there is no exogenous material involved in their formation, and higher blood flow rates that translate to more effective dialysis. However, if an arteriovenous fistula permits very high flow, then excessive “blood steal” can result in inadequate flow to the distal extremities of that limb. This may result in cold extremities of such limb, cramping pains, or tissue damage.

Arteriovenous grafts are much like arteriovenous fistulas, except that an artificial vessel made of a synthetic material is used to join the artery and vein. As such, arteriovenous grafts may result in foreign body reactions. However, arteriovenous grafts can typically be ready for use as a dialysis conduit soon after surgical implantation, unlike arteriovenous fistulas. Arteriovenous grafts are often used when the subject's native vasculature does not permit using an arteriovenous fistula.

### OVERVIEW

While the high blood flow rates of arteriovenous fistulas and grafts are thought to reduce the likelihood of thrombosis, there can be a number of complications including high output heart failure and a distal blood steal syndrome resulting from such flow. In addition, very high flow may result in thrombosis resulting from venous hyperplasia or stenosis occurring either at the graft-vein anastomosis or centrally in the subclavian or axillary veins.

Blood flow restrictors are discussed in this patent document. In some examples, a restrictor apparatus includes a converging entry portion, a diverging exit portion, and optionally a narrowed portion therebetween to restrict the flow of blood through an arteriovenous graft from a subject's artery to a vein. The structure of the restrictor apparatus decreases the pressure and volume of blood flow between the artery and vein to reduce or prevent hyperplasia or stenosis on the venous side, an increased load on the heart, or blood steal, among other things. The restrictor apparatus can be separate from, but couplable to, the arteriovenous graft. The restrictor apparatus can be integral with the arteriovenous graft. In some examples, the restrictor apparatus can be inserted into the arteriovenous graft in a compressed size and shape, and subsequently be allowed to expand to an uncompressed size and shape. Methods of forming and using the restrictor apparatus are also discussed.

In Example 1, an apparatus comprises at least one blood flow restrictor apparatus extending from a first end to a second end, the blood flow restrictor apparatus including: a restrictor entry portion, including a fixed dimensioned convergent first lumen, when implanted, that tapers via a convex radius of curvature of at least about 2 millimeters to substantially match an interior diameter of an arterial portion of an arteriovenous graft at the first end; and a restrictor exit portion, including a fixed dimensioned divergent second lumen, when implanted, that tapers to substantially match an interior diameter of a venous portion of the arteriovenous graft at the second end.

In Example 2, the apparatus of Example 1 optionally comprises a restrictor narrowed portion disposed between the restrictor entry portion and the restrictor exit portion, the restrictor narrowed portion including a fixed, substantially constant dimensioned third lumen connecting the first and second lumens, the third lumen having a smaller interior diameter than at least a portion of the first and second lumens.

In Example 3, the apparatus of Example 2 is optionally configured such that an axial center of the restrictor narrowed portion is located offset from a midpoint of the first and second ends of the blood flow restrictor apparatus.

In Example 4, the apparatus of at least one of Example 2 or 3 is optionally configured such that the fixed, substantially constant dimensioned third lumen is at least about 25 millimeters in length.

In Example 5, the apparatus of at least one of Examples 2-4 is optionally configured such that the interior diameter of the third lumen is at least about 1.5 millimeters.

In Example 6, the apparatus of at least one of Examples 2-5 optionally comprises a biologically active layer on an interior surface of at least a portion of at least one of the first lumen of the restrictor entry portion, the second lumen of the restrictor exit portion, or the third lumen of the restrictor narrowed portion.

In Example 7, the apparatus of at least one of Examples 1-6 optionally comprises the arterial portion of the arteriovenous graft, sized and shaped to be coupled to the restrictor entry portion; and the venous portion of the arteriovenous graft, sized and shaped to be coupled to the restrictor exit portion; wherein the arterial and venous portions of the arteriovenous graft have a substantially similar internal diameter.

In Example 8, the apparatus of Example 7 is optionally configured such that the restrictor apparatus comprises a structure that is separate from, but couplable to, at least one of the arterial portion of the arteriovenous graft or the venous portion of the arteriovenous graft.

In Example 9, the apparatus of Example 8 optionally comprises at least one annular clamp sized and shaped to be disposed around a portion of the arteriovenous graft and a reduced diameter portion of the restrictor apparatus to couple the arteriovenous graft to the at least one restrictor apparatus.

In Example 10, the apparatus of at least one of Examples 1-9 is optionally configured such that the outward taper of the divergent second lumen of the restrictor exit portion includes an exit angle, with respect to a coaxial central axis of the second lumen, of less than or equal to about 6 degrees.

In Example 11, the apparatus of at least one of Examples 1-10 is optionally configured such that the restrictor apparatus is inserted into the arteriovenous graft in a compressed size and shape and assumes an uncompressed size and shape, including the restrictor entry portion and the restrictor exit portion, when secured in an implanted position.

In Example 12, the apparatus of Example 11 is optionally configured such that the restrictor apparatus is biased outward from the compressed size and shape to the uncompressed size and shape.

In Example 13, an apparatus comprises at least one blood flow restrictor apparatus extending from a first end to a second end, the blood flow restrictor apparatus including: a restrictor entry portion, including a fixed dimensioned convergent first lumen, when implanted, that tapers to substantially match an interior diameter of an arteriovenous graft at the first end; and a restriction exit portion, including a fixed dimension divergent second lumen, when implanted, that tapers at an exit angle, with respect to a coaxial central axis of the second lumen, of less than or equal to about 6 degrees to substantially match the interior diameter of the arteriovenous graft.

In Example 14, the apparatus of Example 13 is optionally configured such that the convergent first lumen of the restrictor entry portion includes an entry angle, with respect to a coaxial central axis of the first lumen, of less than or equal to about 6 degrees.

In Example 15, the apparatus of at least one of Example 13 or 14 optionally comprises a restrictor narrowed portion disposed between the restrictor entry portion and the restrictor exit portion, the restrictor narrowed portion including a fixed, substantially constant dimensioned third lumen connecting the first and second lumens.

In Example 16, the apparatus of at least one of Examples 13-15 optionally comprises an arterial portion of the arteriovenous graft, sized and shaped to be coupled to the restrictor entry portion; and a venous portion of the arteriovenous graft, sized and shaped to be coupled to the restrictor exit portion.

In Example 17, the apparatus of at least one of Examples 13-16 is optionally configured such that the restrictor apparatus is inserted into the arteriovenous graft in a compressed sized and shape and assumes an uncompressed size and shape, including the restrictor entry portion and the restrictor exit portion, when secured in an implanted position.

In Example 18, the apparatus of Example 17 is optionally configured such that the restrictor apparatus is biased outward from the compressed size and shape to the uncompressed size and shape.

In Example 19, a method of restricting a flow of blood comprises guiding a converging of the flow of blood from a first fluid lumen defined by a first interior diameter wall of an arteriovenous graft to a second fluid lumen defined by a fixed, substantially constant interior diameter wall of a narrowed portion of at least one restrictor apparatus; and guiding a diverging of the flow of blood from the second fluid lumen defined by the fixed, substantially constant interior diameter wall of the narrowed portion of the restrictor apparatus to a third fluid lumen defined by a second interior diameter wall of the arteriovenous graft; wherein the narrowed portion includes a fixed interior diameter of at least about 1.5 millimeters and a length of at least about 25 millimeters.

In Example 20, the method of Example 19 is optionally configured such that guiding the converging of the flow of blood includes flowing blood over a convex radius of curvature of at least about 2 millimeters.

In Example 21, the method of at least one of Example 19 or 20 optionally comprises inserting an arterial cannula into an arterial end portion of the arteriovenous graft; inserting a venous cannula into a venous end portion of the arteriovenous graft; performing hemodialysis using the arterial and venous cannulas; using the restrictor apparatus located between the arterial and venous cannulas to restrict blood flow bypassing the arterial and venous cannulas through the arteriovenous graft during the hemodialysis; and removing the arterial and venous cannulas from the respective arterial and venous end portions.

In Example 22, the method of Example 21 is optionally configured such that restricting blood flow includes permitting blood flow through the arterial and venous cannulas of at least about 300 cubic centimeters per minute during the hemodialysis.

In Example 23, the method of Example 21 is optionally configured such that restricting blood flow includes permitting blood flow through the arterial and venous cannulas of at least about 400 cubic centimeters per minute during the hemodialysis.

In Example 24, the method of at least one of Examples 19-23 optionally comprises endovascularly inserting the restrictor apparatus in a compressed shape into the arteriovenous graft; and releasing the compressed shape to allow the restrictor apparatus to uncompress.

In Example 25, the method of Example 24 is optionally configured such that endovascularly inserting the restrictor apparatus in the compressed shape includes inserting the restrictor apparatus using a catheter.

In Example 26, the method of at least one of Example 24 or 25 optionally comprises endovascularly inserting a deflated balloon within the restrictor apparatus; inflating the balloon, the balloon including an inflated shape having a first section at a first end and a second section at a second end, the first section being substantially conical and converging from the first end toward the second end, the second section being substantially conical and converging from the second end toward the first end, wherein inflating the balloon within the restrictor apparatus forces the restrictor apparatus to take a shape similar to that of the inflated balloon; deflating the balloon; and removing the balloon from within the restrictor apparatus, the restrictor apparatus maintaining the shape similar to that of the inflated balloon.

In Example 27, the method of at least one of Examples 19-26 optionally comprises endovascularly inserting an outer piece of the restrictor apparatus into the arteriovenous graft, the outer piece having a first diameter and a first length; endovascularly inserting an inner piece of the restrictor apparatus within the outer piece, the inner piece having a shaped inner profile including a convergent first portion that tapers to substantially match the first fluid lumen defined by the first interior diameter wall and a divergent second portion that tapers to substantially match the third fluid lumen defined by the second interior diameter wall; attaching a distal end of the inner piece to a distal end of the outer piece; and attaching a proximal end of the inner piece to a proximal end of the outer piece.

In Example 28, a method comprises endovascularly inserting a compressed shape memory apparatus into an arteriovenous graft; and releasing the shape memory apparatus to allow the shape memory apparatus to uncompress, the uncompressed shape memory apparatus including: an entry portion, including a convergent first lumen portion that tapers to substantially match an interior diameter of an arterial portion of the arteriovenous graft; and an exit portion, including a divergent second lumen portion that tapers to substantially match an interior diameter of a venous portion of the arteriovenous graft.

In Example 29, the method of Example 28 is optionally configured such that endovascularly inserting the compressed shape memory apparatus includes inserting the shape memory apparatus using a catheter.

In Example 30, the method of at least one of Example 28 or 29 is optionally configured such that endovascularly inserting the compressed shape memory apparatus into the arteriovenous graft includes endovascularly inserting a two-piece shape memory apparatus having a first piece including the entry portion and a second piece including the exit portion.

In Example 31, the method of Example 30 optionally comprises endovascularly attaching the first and second pieces of the shape memory apparatus.

In Example 32, the method of Example 31 is optionally configured such that endovascularly attaching the first and second pieces of the shape memory apparatus includes attaching the first piece to the second piece.

In Example 33, the method of at least one of Examples 28-32 is optionally configured such that releasing the shape memory apparatus allows the shape memory apparatus to uncompress, the uncompressed shape memory apparatus including an intermediate portion between the entry portion and the exit portion, the intermediate portion including a substantially cylindrical third lumen portion.

In Example 34, the method of Example 33 is optionally configured such that endovascularly inserting the compressed shape memory apparatus into the arteriovenous graft includes endovascularly inserting a three-piece shape memory apparatus having a first piece including the entry portion, a second piece including the exit portion, and a third piece including the intermediate portion.

In Example 35, the method of Example 34 optionally comprises endovascularly attaching the first, second, and third pieces of the shape memory apparatus.

In Example 36, the method of Example 35 is optionally configured such that endovascularly attaching the first, second, and third pieces of the shape memory apparatus includes magnetically attaching the first, second, and third pieces.

In Example 37, the method of at least one of Examples 28-36 is optionally configured such that endovascularly inserting the compressed shape memory apparatus includes endovascularly inserting a compressed shape memory blood flow restrictor apparatus.

In Example 38, a method comprises endovascularly inserting a deflated balloon and a moldable stent within an arteriovenous graft; inflating the balloon within the moldable stent, the balloon including an inflated shape having a first section at a first end and a second section at a second end, the first section being substantially conical and converging from the first end toward the second end, the second section being substantially conical and converging from the second end toward the first end, wherein inflating the balloon within the moldable stent forces the moldable stent to take a shape similar to that of the inflated balloon; deflating the balloon; and removing the balloon from within the moldable stent, the moldable stent maintaining the shape similar to that of the inflated balloon.

In Example 39, the method of Example 38 is optionally configured such that endovascularly inserting the deflated balloon and the moldable stent includes inserting the deflated balloon and the moldable stent using a catheter.

In Example 40, the method of at least one of Example 38 or 39 optionally comprises inflating the balloon within the moldable stent, the inflated shape of the balloon including a third section between the first section and the second section, the third section being substantially cylindrical.

In Example 41, a method comprises endovascularly inserting an outer piece of a blood flow restrictor apparatus into an arteriovenous graft, the outer piece having a first diameter and a first length; endovascularly inserting an inner piece of the blood flow restrictor apparatus within the outer piece, the inner piece having a shaped inner profile including a convergent first portion that tapers to substantially match an interior diameter of an arterial portion of the arteriovenous graft and a divergent second portion that tapers to substantially match an interior diameter of a venous portion of the arteriovenous graft; attaching a distal end of the inner piece to a distal end of the outer piece; and attaching a proximal end of the inner piece to a proximal end of the outer piece.

In Example 42, the method of Example 41 is optionally configured such that endovascularly inserting the outer and inner pieces includes endovascularly inserting compressed outer and inner pieces of the restrictor apparatus.

In Example 43, the method of Example 42 optionally comprises releasing each of the outer and inner pieces of the restrictor apparatus once inserted to allow each of the outer and inner pieces to uncompress within the arteriovenous graft.

In Example 44, the method of at least one of Examples 41-43 is optionally configured such that endovascularly inserting the outer and inner pieces includes inserting the outer and inner pieces using a catheter.

In Example 45, the method of at least one of Examples 41-44 is optionally configured such that attaching the distal end of the inner piece to the distal end of the outer piece includes attaching an engagement feature of one of inner and outer pieces with a mating engagement feature of the other of the inner and outer pieces.

In Example 46, the method of at least one of Examples 41-45 is optionally configured such that attaching the proximal end of the inner piece to the proximal end of the outer piece includes attaching an engagement feature of one of inner and outer pieces with a mating engagement feature of the other of the inner and outer pieces.

In Example 47, the method of at least one of Examples 41-46 is optionally configured such that endovascularly inserting the inner piece includes the shaped inner profile of the inner piece including a third portion between the first portion and the second portion, the third portion including a substantially cylindrical lumen portion.

In Example 48, a method comprises endovascularly inserting a deflated balloon, a compressed shape memory apparatus, and a moldable stent at a desired endovascular location; releasing the shape memory apparatus to allow the shape memory apparatus to uncompress at portions unconstrained by the moldable stent to form an entry portion and an exit portion; inflating the balloon within the moldable stent and the shape memory apparatus to expand the moldable stent and the shape memory apparatus to form an intermediate portion; deflating the balloon; and removing the balloon from within the moldable stent and the shape memory apparatus, wherein the moldable stent and the shape memory apparatus include: the entry portion, including a convergent first lumen portion that tapers to substantially match an interior diameter of an arterial portion of the arteriovenous graft; the exit portion, including a divergent second lumen portion that tapers to substantially match an interior diameter of a venous portion of the arteriovenous graft; and the intermediate portion between the entry portion and the exit portion, the intermediate portion including a substantially cylindrical third lumen portion.

In Example 49, the method of Example 48 is optionally configured such that endovascularly inserting the deflated balloon, the compressed shape memory apparatus, and the moldable stent includes inserting the deflated balloon, the compressed shape memory apparatus, and the moldable stent using a catheter.

In Example 50, the method of at least one of Example 48 or 49 is optionally configured such that endovascularly inserting the deflated balloon, the compressed shape memory apparatus, and the moldable stent includes endovascularly inserting the deflated balloon, the compressed shape memory apparatus, and the moldable stent within an arteriovenous graft.

In Example 51, an apparatus comprises a compressed shape memory apparatus configured to expand to a desired shape when released; a moldable apparatus sized and shaped to substantially encircle a center portion of the compressed shape memory apparatus to constrain the center portion of the compressed shape memory apparatus; and a deflated balloon sized and shaped to be disposed within the compressed shape memory apparatus and the moldable apparatus, the balloon configured to expand the moldable apparatus to a desired shape with inflation of the balloon.

In Example 52, the apparatus of claim 51 optionally comprises a catheter sized and shaped to house the compressed shape memory apparatus, the moldable apparatus, and the deflated balloon.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various embodiments discussed in the present patent document.

FIG. 1 is a plan view of a hemodialysis system and an environment in which the hemodialysis system can generally be used.

FIG. 2A is a schematic view of an arteriovenous graft and an environment in which the graft can be used, as constructed in accordance with an embodiment.

FIG. 2B is a schematic view of an arteriovenous graft system and an environment in which the graft system can be used, as constructed in accordance with an embodiment.

FIG. 2C is a detailed view of an arteriovenous graft system and an environment in which the graft system can be used, as constructed in accordance with an embodiment.

FIG. 3A is a schematic view of portions of an arteriovenous graft system, as constructed in accordance with an embodiment.

FIG. 3B is a side cross-sectional view along line 3B-3B of FIG. 3A illustrating interior portions of the arteriovenous graft system of FIG. 3A.

FIG. 3C is a transverse cross-sectional view along line 3C-3C of FIG. 3A illustrating the varying diameters of the arteriovenous graft system of FIG. 3A.

FIG. 4A is a schematic view of portions of an arteriovenous graft system, as constructed in accordance with an embodiment.

FIG. 4B is a side cross-sectional view along line 4B-4B of FIG. 4A illustrating interior portions of the arteriovenous graft system of FIG. 4A.

FIG. 4C is a transverse cross-sectional view along line 4C-4C of FIG. 4A illustrating the varying diameters of the arteriovenous graft system of FIG. 4A.

FIGS. 5A-5C illustrate insertion of an example restrictor apparatus into an arteriovenous graft, in accordance with an embodiment.

FIGS. 6A-6B illustrate an example restrictor apparatus, as constructed in accordance with an embodiment.

FIGS. 7A-7C illustrate insertion of an example restrictor apparatus into an arteriovenous graft, in accordance with an embodiment.

FIGS. 8A-8D illustrate insertion of an example restrictor apparatus into an arteriovenous graft, in accordance with an embodiment.

FIGS. 9A-9D illustrate insertion of an example restrictor apparatus into an arteriovenous graft, in accordance with an embodiment.

FIG. 10 is a summary chart from a computer simulation listing blood flow properties when using and not using a restrictor apparatus, as constructed in accordance with an embodiment.

FIG. 11A is a schematic view of a hemodialysis system not including a restrictor apparatus and one or more measurement devices used for in vivo experimentation, as constructed in accordance with an embodiment.

FIG. 11B is a schematic view of a hemodialysis system including a restrictor apparatus and one or more measurement devices used for in vivo experimentation, as constructed in accordance with an embodiment.

FIGS. 11C-11E provide a data chart summarizing in vivo experimentation results of a hemodialysis system including and not including a restrictor apparatus, as constructed in accordance with an embodiment.

FIG. 12 illustrates an example method of forming an arteriovenous graft system, including forming a restrictor apparatus having fixed dimensions.

FIG. 13 illustrates an example method of restricting a flow of blood through an arteriovenous graft system.

### DETAILED DESCRIPTION

Healthy kidneys not only clean blood by filtering out extra water and wastes, but they also produce hormones that help maintain strong bones and healthy blood. When a subject's kidneys fail, numerous debilitating effects are experienced by the subject, including rising blood pressure, accumulation of fluids and toxic wastes in the subject's body and insufficient red blood cell production. Treatment is therefore required to artificially replace the work of the failed kidneys.

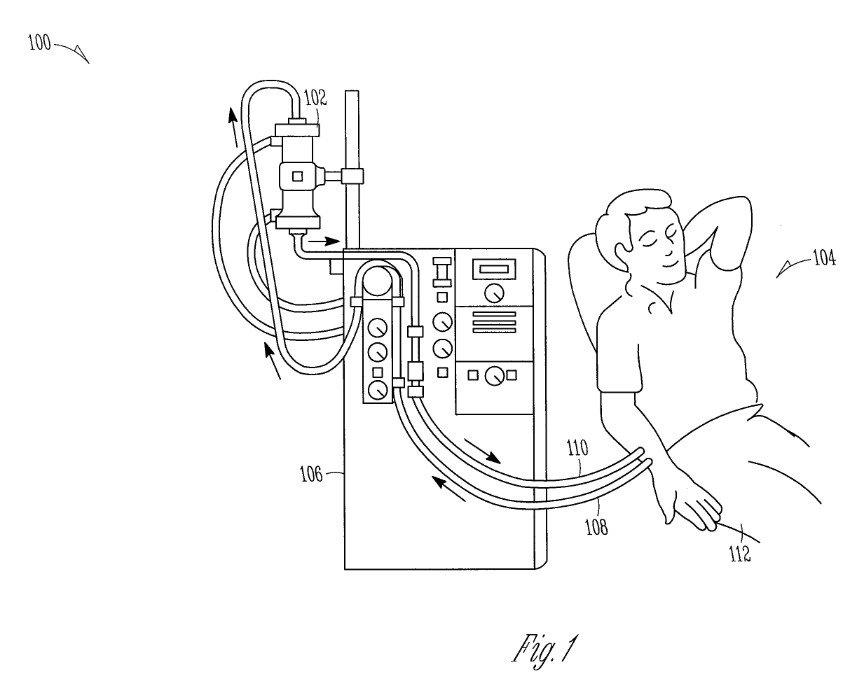

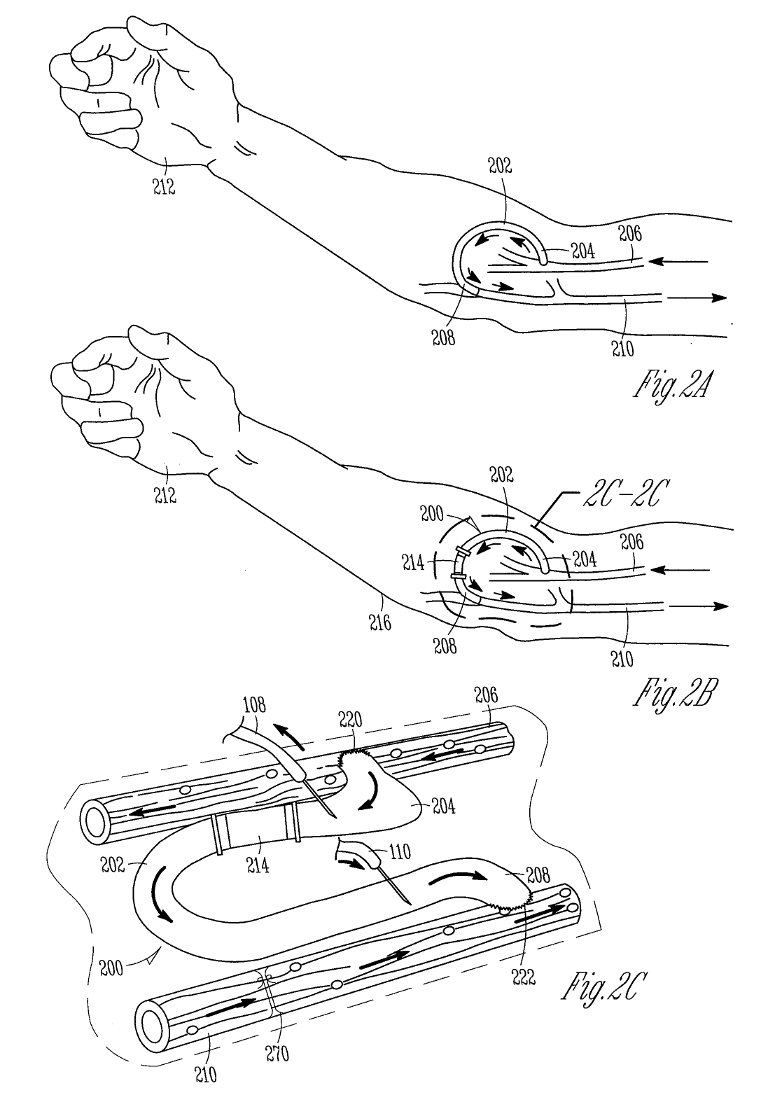

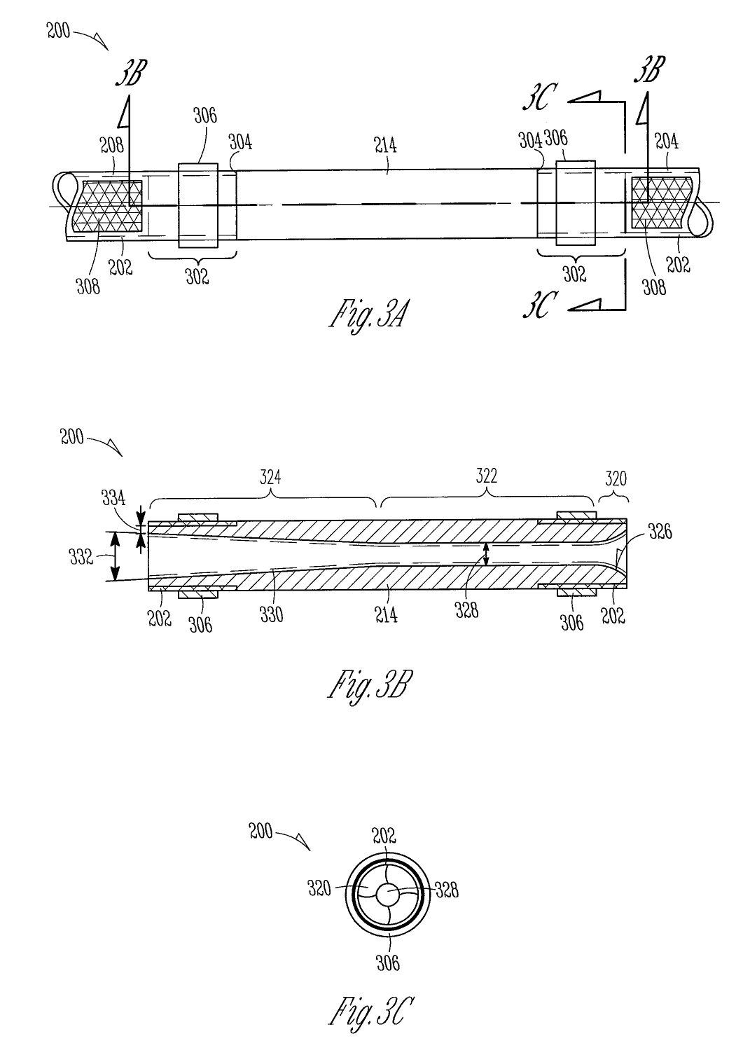

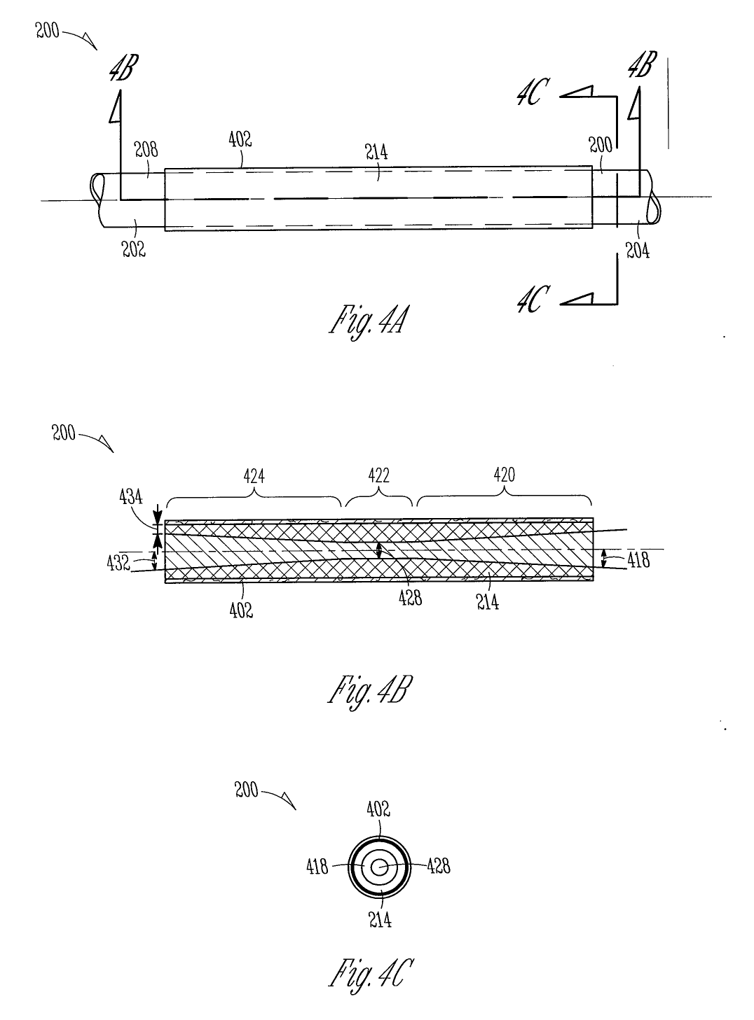

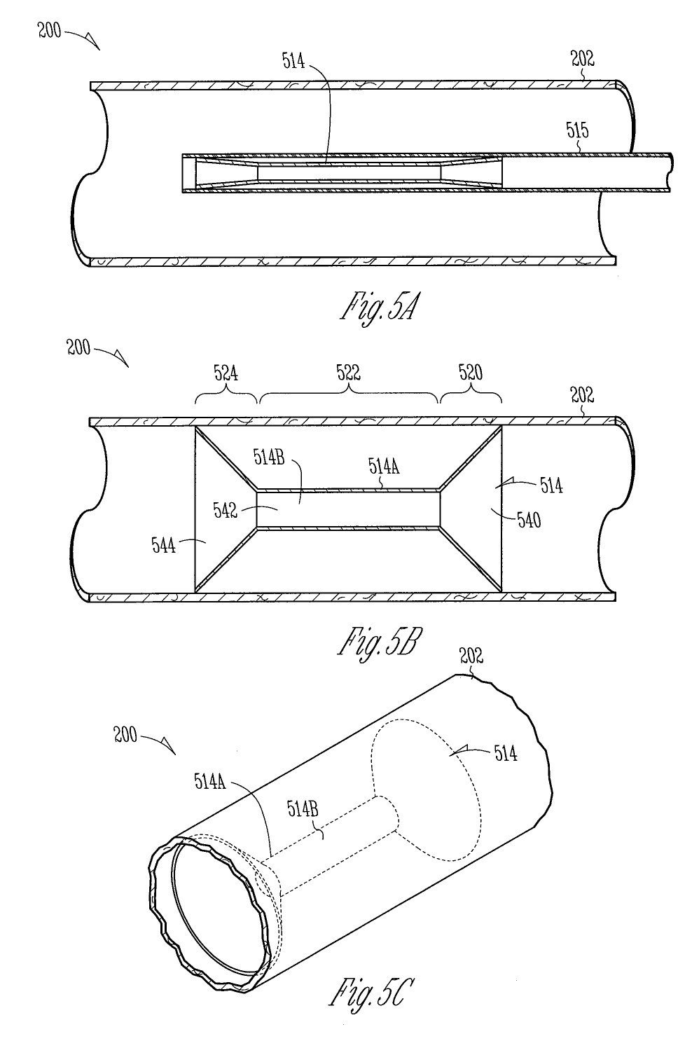

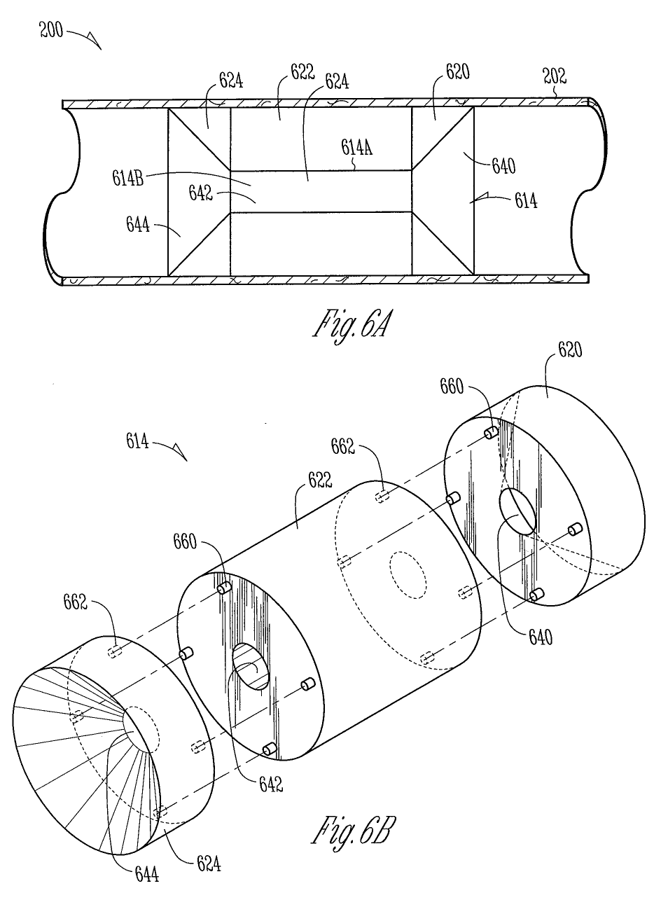

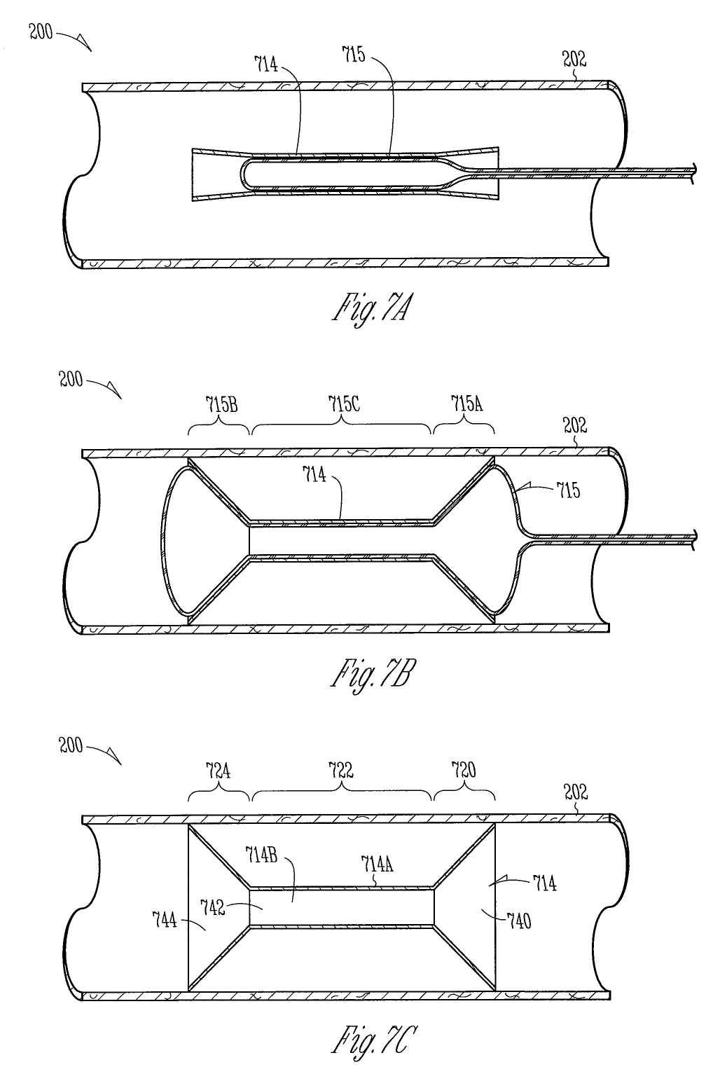

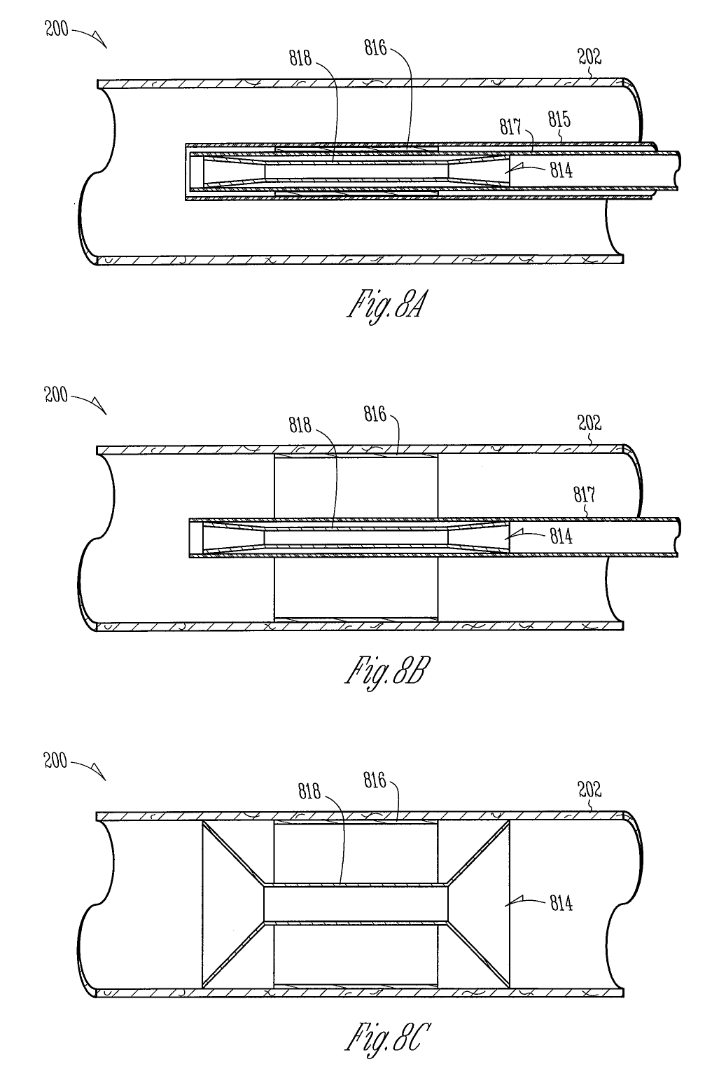

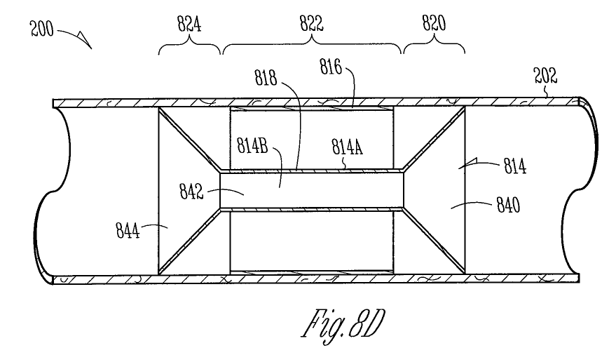

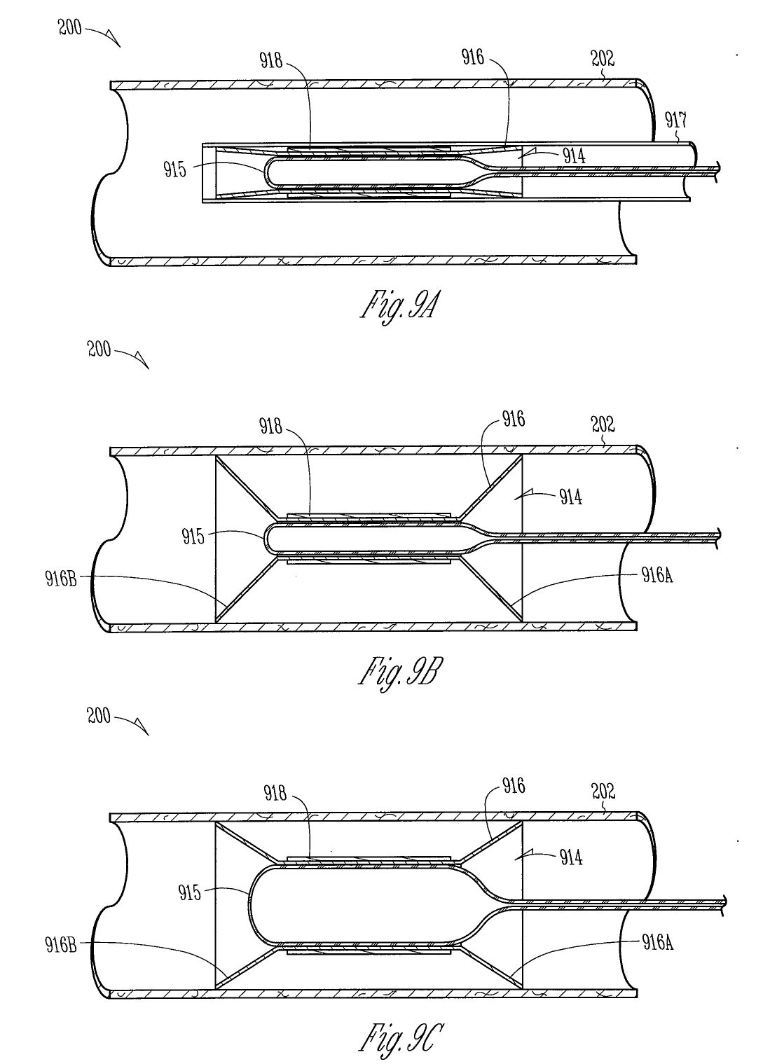

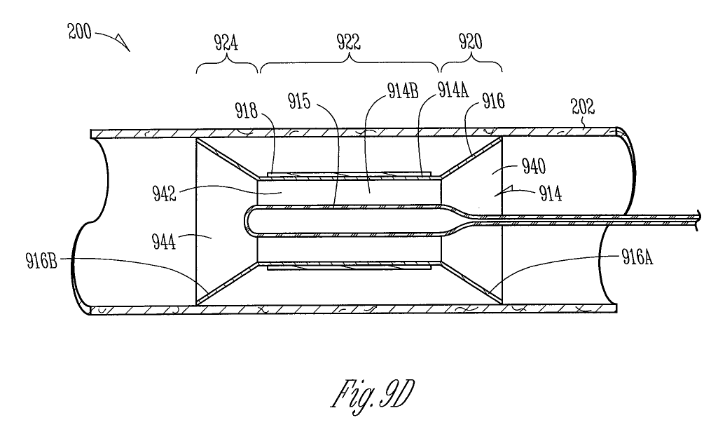

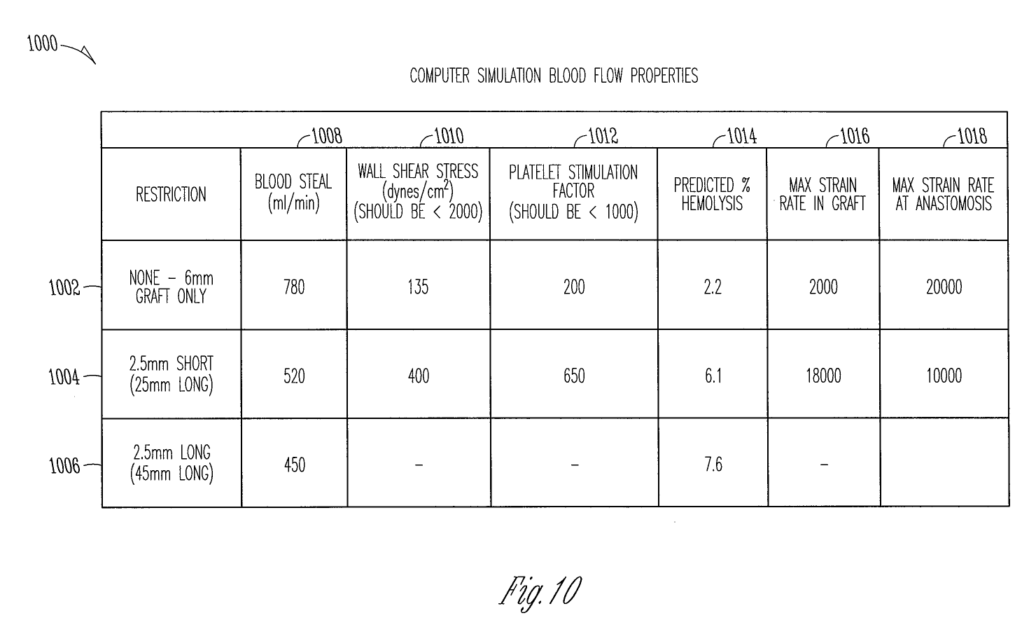

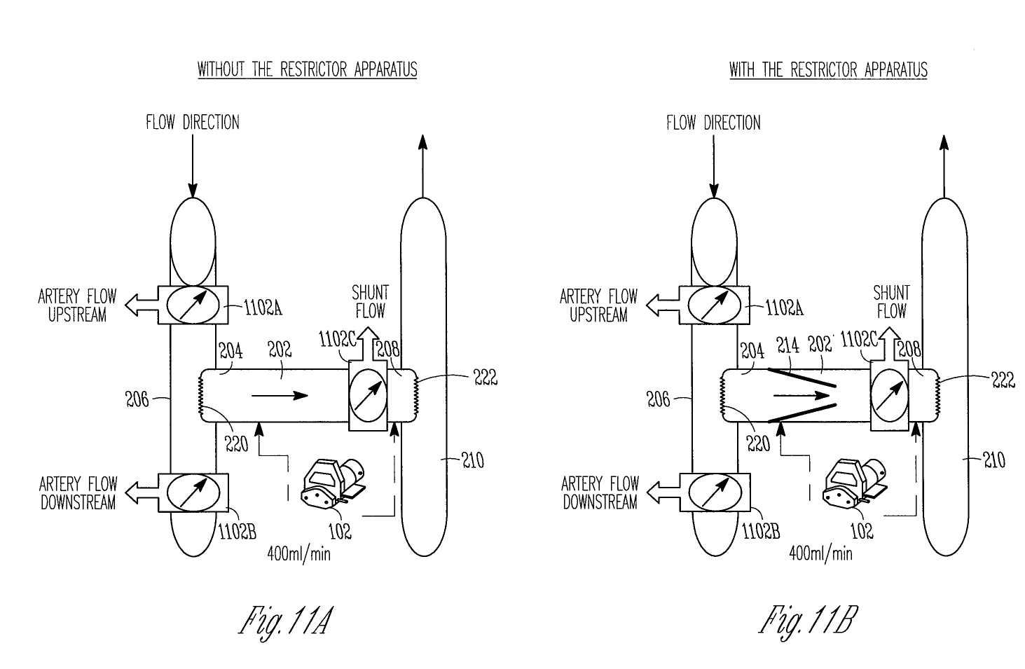

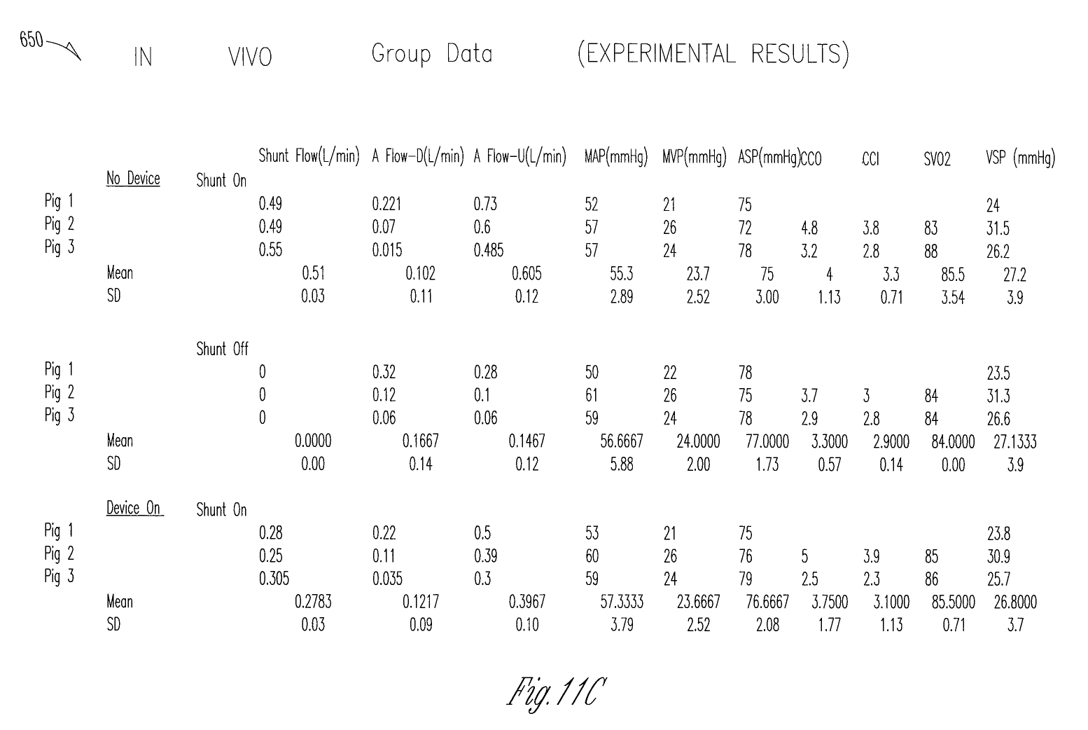

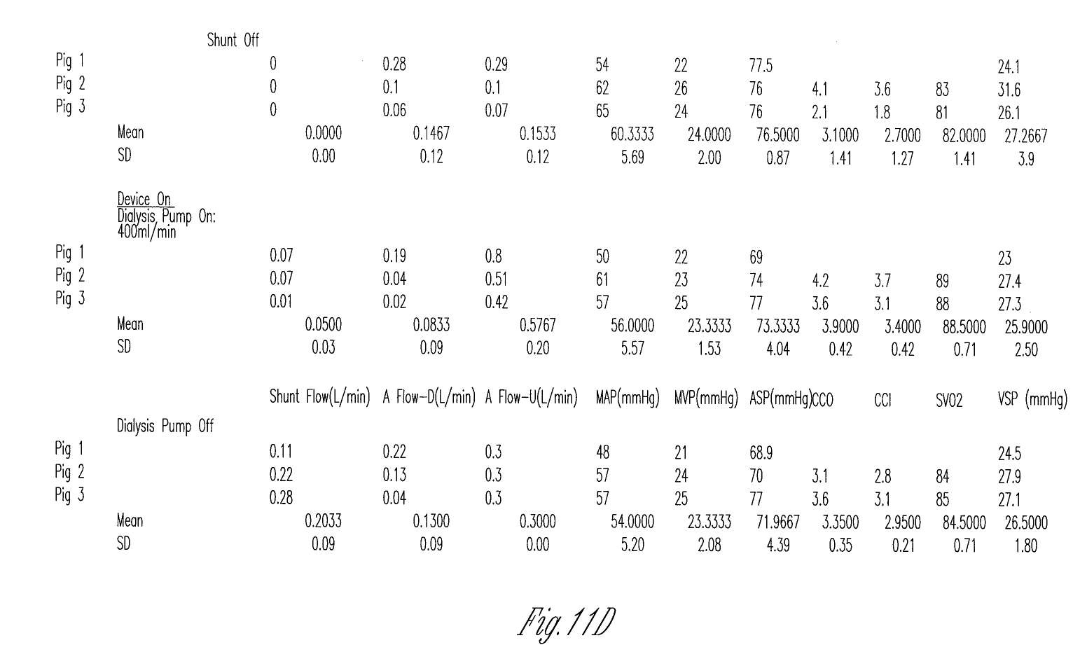

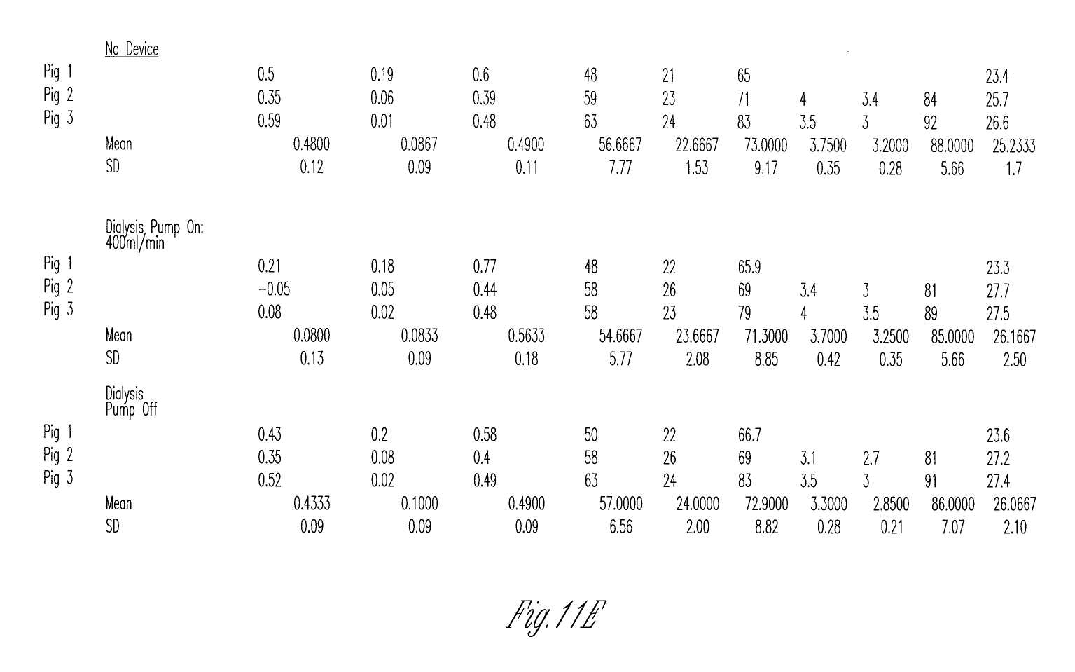

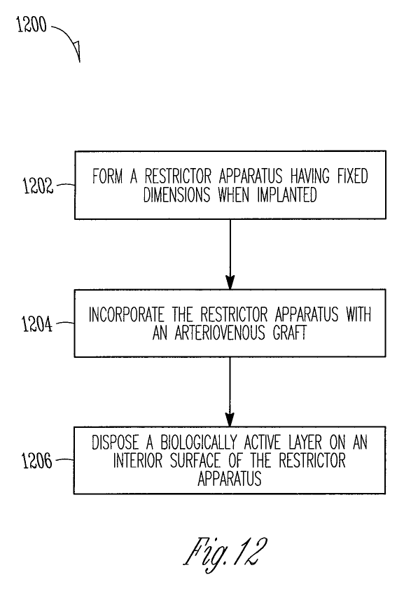

A hemodialysis machine acts as an artificial kidney to remove toxins and water from the subject's blood. Hemodialysis generally uses a special filter, typically a dialyzer 102, to clean the blood. FIG. 1 illustrates a hemodialysis system 100 and a subject 104 with which the hemodialysis system 100 can be used. The hemodialysis system 100, in this example, generally includes a dialysis machine 106, one or more cannulas 108, 110, and an arteriovenous graft 202 (FIG. 2A). As shown in FIG. 2A, the arteriovenous graft 202 extends from an arterial end portion 204, which can be anastomosed with a subject's artery 206, to a venous end portion 208, which can be anastomosed with a subject's vein 210.

As shown in FIG. 2C, an arterial cannula 108 and a venous cannula 110 can be inserted into the arteriovenous graft 202 near the graft-artery anastomosis 220 and graft-vein anastomosis 222, respectively. Then, as shown in FIG. 1, blood from the subject 104 can be drawn via the arterial cannula 108 at the arterial side of the arteriovenous graft and received by the dialysis machine 106 where it is dialyzed (i.e., cleansed). After being dialyzed, the blood can be returned to the subject 104 at the venous side of the arteriovenous graft via the venous cannula 110.

To filter the blood efficiently, the dialysis machine 106 typically requires a blood flow rate of about 400 cubic centimeters per minute (i.e., 400 cc/min). To supply such a high blood flow rate while preventing vessel wall collapse as the dialysis machine 106 extracts blood, a relatively large diameter graft (e.g., a graft about 6 millimeters in tubular interior diameter) is used. However, such large diameter grafts can cause high output heart failure, atrophy of one or more peripheral limbs, such as a hand 212 (FIG. 2A), or thrombosis secondary to venous hyperplasia or stenosis occurring either at the graft-vein anastomosis 222 (FIG. 2C) or centrally in the subclavian or axillary veins.

The present inventors have recognized a need for, among other things, cost-effective vascular access systems, apparatuses, and methods that reduce the excess circulatory load obligated by a relatively large diameter arteriovenous graft 202 and lessen the blood steal of such graft 202 by reducing flow through it, without encouraging clotting, and while still maintaining a high flow rate during dialysis. Accordingly, the present inventors have developed a blood flow restrictor apparatus 214 for use with the arteriovenous graft 202 (collectively referred to as an arteriovenous graft system 200 (see, e.g., FIGS. 2B, 2C, 3A, 3B, 4A, and 4B)). The restrictor apparatus 214 is sized and shaped to, among other things, reduce the basal (non-hemodialysis state) blood flow through the arteriovenous graft 202, while still allowing the flow rates typical for efficient dialysis. In some examples, the restrictor apparatus 214 can, additionally or alternatively, reduce recirculation of dialyzed blood, thereby facilitating the. obtaining of cleaner blood in less time. Reducing recirculation of dialyzed blood increases hemodialysis efficiency, which can lessen the hemodialysis treatment time requirements for subjects 104 with renal failure.

### EXAMPLES

An example of a right arm 216 of a subject 104 (FIG. 1) subcutaneously implanted with an arteriovenous graft system 200 is shown in FIG. 2B. In this example, the arteriovenous graft system 200 includes a tubular or similar arteriovenous graft 202 and an integral or separable blood flow restrictor apparatus 214. The arteriovenous graft system 200 is generally connected between a subject's artery 206, such as one of the brachial, ulnar, or radial arteries, and a subject's vein 210, such as the cephalic vein.

FIG. 2C illustrates in more detail, a portion of the subject's right arm 216 and the arteriovenous graft system 200 subcutaneously implanted therein. The arteriovenous graft system 200 provides a shunted path of low blood flow resistance that allows a substantial portion of the arterial blood flowing through the subject's artery 206 to be diverted at the graft-artery sewn anastomosis 220, through the arteriovenous graft 202 and restrictor apparatus 214, to the subject's vein 210 at the graft-vein sewn anastomosis 222, such as during the blood diversion of a hemodialysis session.

During hemodialysis, an arterial cannula 108 and a venous cannula 110 are inserted into the arteriovenous graft 202 near the graft-artery anastomosis 220 and the graft-vein anastomosis 222, respectively. Blood is drawn from the subject 104 (FIG. 1) upstream of the restrictor apparatus 214 via the arterial cannula 108 at the arterial end portion 204 of the arteriovenous graft 202, sent through a dialysis machine 106 (FIG. 1) where it is dialyzed, and returned to the subject 104 downstream of the restrictor apparatus 214 at the venous end portion 208 of the arteriovenous graft 202 via the venous cannula 110. In one example, but as may vary, the venous cannula 110 and the arterial cannula 108 are inserted into the subject's skin about 2-3 centimeters or more apart, which translates to about 8-10 centimeters or more separation on the arteriovenous graft 202 due to a U-shape implantation configured, such as is shown in FIG. 2C. This 8-10 centimeters or more separation reduces or prevents recirculation of dialyzed blood through the arteriovenous graft 202. The blood flow restrictor apparatus 214 can be placed or located in the arteriovenous graft 202 between such insertion points of the arterial cannula 108 and the venous cannula 110. The blood flow restrictor apparatus 214 permits the requisite high flow from the arterial cannula 108 and through the venous cannula 110 during dialysis, but restricts the blood flow through the fixed dimensions of the restrictor apparatus itself, during and between hemodialysis sessions. This reduces the complications associated with a high flow rate arteriovenous graft, such as high output heart failure, atrophy of the distal hand 212, or thrombosis secondary to venous hyperplasia or stenosis occurring either at the graft-vein anastomosis 222 or centrally in the subclavian or axillary veins, as discussed above.

To prevent insertion of one or both of the arterial 108 or venous 110 cannula into the restrictor apparatus 214, the restrictor apparatus 214 itself can include a non-puncturable structure (see, e.g., FIGS. 3A-3B) or a rigid collar 402 or other puncture resistant covering can be disposed around an exterior of the restrictor apparatus 214 (see, e.g., FIGS. 4A-4B).

In certain examples, the arteriovenous graft 202 includes a tubular structure composed of or including a synthetic material, such as GORTEX™ manufactured by W.L. Gore & Associates, Inc. of Newark, Del. Additionally or alternatively, the arteriovenous graft 202 can include a woven or other self-sealing material made of any of a variety of one or more biocompatible materials, including biocompatible polymers, metals, alloys, or a combination thereof, such as polyester, polytetrafluoroethylene, polyethylene, polypropylene, polyurethane, silicone, stainless steel, titanium, or platinum, some of which are manufactured by Gish Biomedical, Inc. of Rancho Santa Margarita, Calif.

The human body may react to introduction of the synthetic materials of an arteriovenous graft 202. The body's reaction may include thrombus formation in or around the arteriovenous graft 202. While woven graft materials, such as GORTEX™, may not be recognized by the subject's body as a foreign body to the same degree as non-woven materials, woven materials may still experience some degree of body reaction, such as inflammation. For this reason, the arteriovenous grafts 202 can be made larger in interior diameter than what is needed to accommodate the dialysis machine's 106 (FIG. 1) about 400 cc/min requisite blood flow. This larger size, in turn, can result in high volume blood flow (e.g., 800-900 cc/min), which may further result in hyperplasia, among other things. Hyperplasia is a condition that may occur when the higher pressure/volume of the arterial flow crosses the boundary from the relatively non-compliant arteriovenous graft 202 to the more compliant outflow vein 210 at the venous anastomosis 222. The resultant intimae hyperplasia in the vein 210 adjacent to the anastomosis 222 may lead to progressive stenosis and eventually premature clotting and arteriovenous graft 202 occlusion. In addition to hyperplasia and stenosis, the large obligate shunted blood volumes may lead to an increased load on the heart and blood steal that results in poor circulation at the extremity beyond or distal to the arteriovenous graft 202.

The restrictor apparatus 214 comprises a size and shape that reduces the pressure and volume of blood flow through the arteriovenous graft 202 (e.g., by about 40-50%) generally without thrombus formation, and accordingly may reduce or eliminate the above discussed problems with hyperplasia, stenosis, increased heart load, or blood steal. Further, the restrictor apparatus 214 still allows adequate blood flow typically needed by the dialysis machine 106 during dialysis sessions (e.g., about 400 cc/min blood flow; however, in certain circumstances about 300 cc/min may suffice). In certain examples, but not by way of limitation, the arteriovenous graft 202 is about 5-6 inches long and about 6 millimeters in interior diameter outside the region of the restrictor apparatus 214. As shown, the implanted shape of the arteriovenous graft 202 between the subject's artery 206 and vein 210 can generally resemble a U-shape (i.e., make an approximate 180 degree change in direction). In one such example, the restrictor apparatus 214 is disposed on a generally straight leg portion of the U-shape. In another example, the restrictor apparatus 214 comprises a pliable (i.e., bendable) material and is disposed on a curved portion of the U-shape. As phantomly shown, the subject's vein 210 can be ligated 270 upstream of the graft-vein anastomosis 222.

Although the present examples focus on an arteriovenous graft system 200 subcutaneously implanted within a subject's arm 216 (see, e.g., FIG. 2B), the present subject matter is not so limited. The arteriovenous graft system 200 can alternatively be implanted in any suitable location of the subject's body 104 (FIG. 1). For instance, in certain examples, the arteriovenous graft system 200 can be implanted within a subject's leg 112 (FIG. 1).

FIG. 3A illustrates portions of an example of an arteriovenous graft system 200. The arteriovenous graft system 200 comprises an arteriovenous graft 202 and a restrictor apparatus 214. As shown, the restrictor apparatus 214 can comprise a structure separate from, but couplable to, the arteriovenous graft 202. In certain examples, the arteriovenous graft 202 comprises a tubular structure having an arterial end portion 204 and a venous end portion 208. The restrictor apparatus 214 can be interposed between the arterial 204 and venous 208 end portions and coupled to adjacent tubular arteriovenous graft 202 portions via reduced apparatus diameter portions 302. The reduced apparatus diameter portions 302 create a shoulder 304 on the restrictor apparatus 214 to which the arterial 204 and venous 208 end portions can abut against when the tubular graft portions 204, 208 are fitted over the reduced apparatus diameter portions 302. The arteriovenous graft 202 and the restrictor apparatus 214 can be securely coupled to one another via stainless steel clamps 306, such as those manufactured by Oetiker, Inc. of Marlette, Mich. Advantageously, clamp materials such as stainless steel and the like are durable, non-corrosive, and non-thrombogenic.

As discussed above, blood from the subject 104 (FIG. 1) flows from an artery 206 (FIG. 2C), through the shunted arteriovenous graft 202 and restrictor apparatus 214, and into a vein 210 (FIG. 2C). To connect the subject 104 to a dialysis machine 106, an arterial 108 and a venous 110 cannula (FIG. 2C) are inserted through the skin and into the arteriovenous graft 202. Blood is removed from the subject 104 through the arterial cannula 108, circulated through the dialysis machine 106, and returned to the subject 104 through the venous cannula 110. In certain examples, the arteriovenous graft 202 comprises a woven material 308 configured to be punctured by the cannulas 108, 110 and to self-seal upon their removal. In other examples, the arteriovenous graft 202 can include dedicated cannula injection portions, which include a self-sealing material, such as silicone or the like.

FIG. 3B is a side cross-sectional view taken along line 3B-3B of FIG. 3A and illustrates the interior structure of one example of an arteriovenous graft system 200. The arteriovenous graft system 200, according to this example, includes an arteriovenous graft 202 coupled to an intermediately disposed restrictor apparatus 214. The arteriovenous graft 202 is securely coupled to the restrictor apparatus 214 via one or more annular clamps 306, such as stainless-steel annular clamps. As shown, but as may vary, the restrictor apparatus 214 can include a side cross-sectional profile having three portions including a restrictor entry portion 320, a restrictor narrowed portion 322, and a restrictor exit portion 324. In another example, the restrictor apparatus 214 can include a side cross-sectional profile having two portions including a restrictor entry portion 320 and a restrictor exit portion 324. Each of the restrictor entry portion 320, the restrictor narrowed portion 322, and the restrictor exit portion 324, if present, have specified fixed internal dimensions (i.e., interior diameters and longitudinal lengths) based on one or more desired blood flow characteristics. Like most foreign objects introduced into a subject's body, it is advantageous to keep the exterior size of the restrictor apparatus 214 small.

In this example, the interior structure of the restrictor apparatus 214 includes a restrictor entry portion 320 having a radius of curvature, a constant diameter restrictor narrowed portion 322, and a gently tapered diverging restrictor exit portion 324. It is desirable to have a smooth transition between the arteriovenous graft 202 and the restrictor apparatus 214. A restrictor entry portion 320 having a large entry radius 326 reduces turbulence, which causes platelets in the blood to collide, and which can induce clot formation. To reduce or avoid turbulent blood flow, varying examples of the restrictor apparatus 214 comprise an entry having a radius of curvature of about 2 millimeters or more. As shown, the restrictor entry portion 320 tapers from (1) a diameter substantially similar to an interior diameter of the arteriovenous graft 202 on a first end of the restrictor entry portion 320 to (2) the diameter of the restrictor narrowed portion 322 on a second end of the restrictor entry portion 320.

The restrictor narrowed portion 322 is generally smooth and generally maintains a fixed and constant diameter 328 along its length. The generally smooth finish of the restrictor narrowed portion 322 helps to prevent thrombosis by not encouraging turbulent blood flow. A longer restrictor narrowed portion 322 will generally further reduce blood flow, but should not be so long as to encourage clotting. In certain examples, the restrictor narrowed portion 322 includes a length of between 1-100 millimeters, such as at least about 25 millimeters. In certain examples, the effective interior diameter of the restrictor narrowed portion 322 is at least about 1.5 millimeters. In certain other examples, the effective interior diameter of the restrictor narrowed portion 322 is at least about 2.5 millimeters, which is believed to stop high viscous shear rates and to successfully reduce the flow of blood through the arteriovenous graft system 200.

To inhibit thrombus formation, the restrictor apparatus 214 can comprise a coating of a biologically active layer 330 (e.g., an anti-thrombogenic coating), such as that manufactured by Carmeda of Upplands Vasby, Sweden, which effectively reduces the interior diameter 328 of the restrictor narrowed portion 322. Thus, in certain examples, the pre-coating interior diameter 328 of the restrictor narrowed portion 322 is about 2.8-3.0 millimeters, such that when the biologically active layer 330 is taken into account, the effective interior diameter 328 of the restrictor narrowed portion is about 2.5 millimeters or more. The biologically active layer 330 can be applied to the surface of the restrictor narrowed portion 322 by coating, spraying, dipping, or vapor deposition. Such layer 330 can extend along the linear length as phantomly shown in FIG. 3B, or be localized to a particular area.

The restrictor exit portion 324 is shown gently tapered having an exit angle 332. Computer simulation indicates that an exit angle 332 of about 6 degrees or less advantageously inhibits or prevents blood flow separation or flow turbulence. As shown, the restrictor exit portion 324 diverges from the diameter 328 of the restrictor narrowed portion 322 on a first end to a diameter that is substantially similar to the interior diameter of the arteriovenous graft 202 on a second end. In certain examples, a step 334 of about 0.5 millimeters or less can exist at the exit of the restrictor apparatus 214 so that there is essentially no discontinuity between the exit portion 324 of the restrictor and the interior diameter of the arteriovenous graft 202.

Together, in at least one example, the restrictor entry portion 320, the restrictor narrowed portion 322, and the restrictor exit portion 324 decrease the dynamic pressure and volume of blood flow passing through the arteriovenous graft system 200. This lessens the blood steal from a limb 212 (FIG. 2B) peripheral to the arteriovenous graft system 200 and reduces the blood flow loads on the heart and veins, all without affecting needed dialysis flow rates and without encouraging clotting. The amount of flow restriction provided by the restrictor apparatus 214 is dependent on the interior diameter and length of the apparatus, such as the interior diameter and length of the restrictor narrowed portion 322. For instance, a longer restrictor narrowed portion 322 generally results in greater flow restriction, but may result in clotting if too long. On the other hand, a shorter restrictor narrowed portion 322 generally results in less flow restriction and can therefore be less effective in reducing blood steal (see, e.g., FIG. 5). A greater restrictor narrowed portion diameter 328 generally results in less clotting, but also less restriction and more blood steal. Advantageously, the separate structure restrictor apparatus 214 illustrated in FIGS. 3A-3C can be used with a conventional vascular access graft, such as by retrofitting the restrictor apparatus 214 into an intermediate portion of an existing arteriovenous graft 202 that has been cut into two pieces. Alternatively, the separate structure restrictor apparatus 214 can be disposed (e.g., slid) within a conventional vascular access graft.

FIG. 3C is a transverse cross-section along line 3C-3C of FIG. 3A and illustrates the varying diameters of one example of an arteriovenous graft system 200. Taken at an outermost end of a reduced diameter portion 302 (FIG. 3A), the cross-section shown in FIG. 3C shows an annular clamp 306 encircling a tubular arteriovenous graft 202 and the tapered restrictor entry portion 320. As shown, the restrictor entry portion 320 tapers to an interior diameter 328 of the restrictor narrowed portion 322. While FIGS. 3A-3C illustrate a traverse cross-section of the arteriovenous graft system 200 having a circular configuration, the traverse cross-section can also be oval or some other configuration.

FIG. 4A illustrates portions of another example of an arteriovenous graft system 200. In this example, the arteriovenous graft system 200 comprises an arteriovenous graft 202 and an integral restrictor apparatus 214. Unlike the restrictor apparatus 214 of FIGS. 3A-3C, the restrictor apparatus 214 of FIGS. 4A-4C together with the arteriovenous graft 202 comprise a unitary construction. The restrictor apparatus 214 can be encircled or surrounded, at least in part, by a relatively non-penetrable (i.e., non-puncturable) collar 402. This prevents cannula 108, 110 (FIG. 2C) insertions into the restrictor apparatus and helps permit a caregiver to be able to palpate the restrictor to determine its position. In certain examples, the collar 402 comprises a rigid biocompatible material, such as a biocompatible metal (e.g., titanium or stainless-steel) or a biocompatible plastic.

FIG. 4B is a side cross-sectional view taken along line 4B-4B of FIG. 4A and illustrates the interior structure of another example of an arteriovenous graft system 200. The arteriovenous graft system 200, in this example, includes an arteriovenous graft 202 integrated with a restrictor apparatus 214. As shown, the restrictor apparatus 214 can include a side cross-sectional profile that includes a restrictor entry portion 420, a restrictor narrowed portion 422, and a restrictor exit portion 424. Each of the restrictor entry portion 420, the restrictor narrowed portion 422, and the restrictor exit portion 424 have specified fixed internal dimensions (i.e., interior diameters and longitudinal lengths), which can be established based on one or more desired blood flow characteristics. For instance, the arteriovenous graft system 200 can include varying interior dimensions in the vicinity of the restrictor apparatus 214 such that the walls are thicker at the restrictor entry portion 420, the restrictor narrowed portion 422, and the restrictor exit portion 424 than at the arterial 204 and venous 208 end portions of the arteriovenous graft 202 (FIG. 4A).

In this example, the interior structure of the restrictor apparatus 214 includes a gently tapered converging restrictor entry portion 420, a constant diameter restrictor narrowed portion 422, and a gently tapered diverging restrictor exit portion 424. It is believed to be desirable to have a smooth transition between the interior diameter of the arteriovenous graft 202 and that of the restrictor apparatus 214. A restrictor entry portion 420 having as large (or near as large) as entry radius 326 (FIG. 3B) as possible may reduce turbulence, which causes platelets in the blood to collide and may induce clot formation. To avoid turbulent blood flow, in certain examples, the restrictor apparatus 214 includes an entry having a radius of curvature of at least about 2 millimeters. As another example, FIG. 4B shows an example in which the restrictor entry portion 420 can include a converging tapered entry angle 418 of about 6 degrees or less.

The restrictor narrowed portion 422 is generally smooth and maintains a fixed and constant diameter 428 along its length. The generally smooth finish of the restrictor narrowed portion 422 helps to prevent thrombosis by not encouraging turbulent blood flow. A longer restrictor narrowed portion 422 further reduces blood flow; however, the restrictor narrowed portion 422 should not be so long as to reduce flow to an extent that encourages clotting. In certain examples, the restrictor narrowed portion 422 comprises a length between 1-100 millimeters, such as at least about 25 millimeters. In certain examples, the effective interior diameter of the restrictor narrowed portion 322 is at least about 1.5 millimeters. In certain other examples, the effective interior diameter of the restrictor narrowed portion 322 is at least about 2.5 millimeters, which is expected to stop high viscous shear rates and to successfully reduce the flow of blood through the arteriovenous graft system 200.

The restrictor exit portion 424 is shown gently tapered having an exit angle 432. An exit angle 432 of about 6 degrees or less advantageously prevents blood flow separation and flow turbulence. As shown, the restrictor exit portion 424 diverges from the diameter 428 of the restrictor narrowed portion 422 on a first end to a diameter substantially similar to the interior diameter of the arteriovenous graft 202 on a second end. A step 434 of about 0.5 millimeters or less can exist at the exit of the restrictor apparatus 214 so that there is essentially no discontinuity between the restrictor and the interior diameter of the arteriovenous graft 202.

In certain examples, the restrictor entry portion 420, the restrictor narrowed portion 422, and the restrictor exit portion 424 decrease the dynamic pressure and volume of blood flow passing through the arteriovenous graft system 200. This lessens the blood steal from a peripheral limb 212 (FIG. 2B) and reduces the blood flow load on the heart and veins, all without affecting needed dialysis flow rates and without encouraging clotting. The amount of flow restriction provided by the restrictor apparatus 214 depends on its interior diameter and length, such as the interior diameter and length of the restrictor narrowed portion 322. For instance, a longer narrowed portion 422 will further reduce flow, but may result in clotting if too long. A greater diameter 428 of the narrowed portion will result in less clotting, but also less flow restriction.

FIG. 4C is a transverse cross-section along line 4C-4C of FIG. 4A and illustrates the varying diameters of one example of an arteriovenous graft system 200. Taken at an end of the restrictor apparatus 214, the cross-section shown in FIG. 4C shows a collar 402 about the walls of the restrictor apparatus 214 and the tapered restrictor entry portion 420. As shown, the restrictor entry portion 420 tapers to an interior diameter 428 of the restrictor narrowed portion 422. While FIGS. 4A-4C illustrate a traverse cross-section of the arteriovenous graft system 200 having a circular configuration, the traverse cross-section can also be oval or some other configuration.

In some examples, a restrictor apparatus 214 can be inserted within an existing arteriovenous graft 202, which is already implanted within a subject's body. In some examples, the restrictor apparatuses 214 described herein can be inserted at desired endovascular locations other than within an arteriovenous graft 202.

For instance, FIGS. 5A-5C illustrate an example of insertion of a shape memory restrictor apparatus 514 into an arteriovenous graft 202. The “shape memory” property permits the apparatus 514 to “remember” a previous shape. For example, the shape memory restrictor apparatus 514 can be compressed or otherwise deformed (e.g., compressed within a sleeve), and can then return toward or regain its pre-deformation shape when uncompressed or otherwise released (e.g., when the sleeve is removed). The shape memory apparatus 514 can include a generally tubular wall 514A defining a lumen 514B therethrough.

In some examples, a compressed shape memory restrictor apparatus 514 can be endovascularly inserted into the already-implanted arteriovenous graft 202. In certain examples, the shape memory restrictor apparatus 514 is stent-like in configuration. In some examples, the shape memory restrictor apparatus 514 can be formed from one or more materials including, but not limited to, a shape memory metal, such as Nitinol. In some examples, the shape memory restrictor apparatus 514 can include a substantially impermeable coating, membrane, or other material, such as, for instance, Dacron or polytetrafluoroethylene (PTFE). The substantially impermeable material, in some examples, can stretch when the shape memory restrictor apparatus 514 is expanded, as described herein, to define a substantially fluid impermeable wall to perform blood flow restriction, as described herein. The shape memory restrictor apparatus 514 can include a shape memory metal with a substantially impermeable material coating, sheath, or surface, such as to inhibit or prevent blood or other fluids from passing through the generally tubular wall 514A of the shape memory restrictor apparatus 514.

In an example, the compressed shape memory restrictor apparatus 514 can be compressed within a retractable sleeve 515, such as for delivery to a desired location. In an example, the compressed shape memory restrictor apparatus 514 can be delivered to a location within the implanted arteriovenous graft 202, such as by using an intravascular delivery catheter. Once at the desired implant location, the shape memory restrictor apparatus 514 can be released, such as to allow the shape memory restrictor apparatus 514 to uncompress and take a desired implanted shape within the arteriovenous graft 202. In an example, the shape memory restrictor apparatus 514 can be released, such as by retracting the retractable sleeve 515 and allowing the shape memory restrictor apparatus 514 to assume the desired shape. In an example, the shape memory restrictor apparatus 514 is capable of expanding to a maximum diameter that is larger than a diameter of the arteriovenous graft 202. This creates a frictional engagement of the outer diameter of the shape memory restrictor apparatus 514 and the inner diameter of the arteriovenous graft 202 when the shape memory restrictor apparatus 514 is released therein. In an example, the shape memory restrictor apparatus 514 is capable of expanding to a non-constricting maximum diameter in which a diameter of the ends of the restrictor apparatus 514 are larger than a diameter of the arteriovenous graft 202 when the restrictor apparatus 514 is unconstrained. This can be used to create a frictional engagement of the outer diameter of the ends of the shape memory restrictor apparatus 514 and the inner diameter of the arteriovenous graft 202 when the restrictor apparatus 514 is released therein.

In an example, the uncompressed shape memory restrictor apparatus 514 can include an entry portion 520. The entry portion 520 can include a convergent first lumen portion 540 that tapers to substantially match an interior diameter of an arterial portion of the arteriovenous graft 202. In an example, the uncompressed shape memory restrictor apparatus 514 includes an exit portion 524. The exit portion 524 can include a divergent second lumen portion 544 that tapers to substantially match an interior diameter of a venous portion of the arteriovenous graft 202. In an example, the uncompressed shape memory restrictor apparatus 514 can include an intermediate portion 522 between the entry portion 520 and the exit portion 524. In an example, the intermediate portion 522 can include a substantially cylindrical third lumen portion 542.

FIGS. 6A and 6B illustrate an example shape memory restrictor apparatus 614, in accordance with an embodiment. When implanted, in an example, the shape memory restrictor apparatus 614 can be generally similar to the shape memory restrictor apparatus 514 described above. However, in some examples, the shape memory restrictor apparatus 614 can include more than one piece. The pieces can be inserted separately and attached within the subject, or the pieces can be fused or otherwise attached together before implantation and then implanted. The shape memory restrictor apparatus 614, in some examples, can include a shape memory blood restrictor apparatus 614 for implantation within an arteriovenous graft 202. In some examples, the shape memory restrictor apparatus 614 can be formed from similar materials as those described above with respect to the shape memory restrictor apparatus 514.

In some examples, the shape memory restrictor apparatus 614 can include a two-piece shape memory restrictor apparatus 614, such as having a first piece 620 including an entry portion and a second piece 624 including an exit portion. In some examples, the shape memory restrictor apparatus 614 can include a third piece 622, such as including an intermediate portion. In some examples, the shape memory restrictor apparatus 614 can include pieces in addition to (e.g., and in accordance with) the first, second, and third pieces 620, 624, 622 described herein.

In an example, the pieces of the shape memory restrictor apparatus 614 can be individually compressed and retained within one or more retractable sleeves for endovascular (for instance, intravenous) insertion into the arteriovenous graft 202. The pieces of the shape memory restrictor apparatus 614 can each be retained within a separate retractable sleeve, or two or more pieces can be retained in a single retractable sleeve. The pieces of the shape memory restrictor apparatus 614 can then be endovascularly inserted into the implanted arteriovenous graft 202, for instance, using a single delivery catheter or other delivery technique. In some examples, at least two of the pieces of the shape memory restrictor apparatus 614 can be endovascularly inserted into the arteriovenous graft 202 using different delivery catheters. Once inserted within the arteriovenous graft 202, the sleeves can be retracted, such as to deploy and permit decompression of each of the pieces of the shape memory apparatus restrictor 614. In an example, mating or other engagement features 660, 662, such as generally depicted in FIG. 6B, can be engaged to each other to attach the pieces of the shape memory restrictor apparatus 614. In some examples, the engagement features 660, 662 can include, but are not limited to, one or more mating hooks or clasps, magnets, mating detents, pins, docking mechanisms, or adhesive surfaces. In an example, the shape memory restrictor apparatus 614 need not include any engagement features; the pieces of the shape memory restrictor apparatus 614 can be held together through mutual and adjacent frictional engagement with the arteriovenous graft 202 when deployed. In an example, the pieces of the shape memory restrictor apparatus 614 can be fused together or otherwise attached before implantation, and then compressed and endovascularly inserted within the arteriovenous graft 202, such as by using a retractable sleeve and delivery catheter or another delivery technique.

Once deployed in the desired location within the arteriovenous graft 202, in some examples, the pieces of the shape memory restrictor apparatus 614 can take desired shapes (or “remember” and return toward their intended shapes). In an example, the uncompressed first piece 620 forms an entry portion of the shape memory restrictor apparatus 614 that includes a convergent first lumen 640 that tapers to substantially match an interior diameter of an arterial portion of the arteriovenous graft 202. In an example, the uncompressed second piece 624 forms an exit portion of the shape memory restrictor apparatus 614 that includes a divergent second lumen 644 that tapers to substantially match an interior diameter of a venous portion of the arteriovenous graft 202. In an example, the uncompressed third piece 622 forms an intermediate portion of the shape memory restrictor apparatus 614 between the first piece 620 and the second piece 624. In an example, the third piece 622 can include a substantially cylindrical third lumen 642. When attached, the pieces of the shape memory restrictor apparatus 614 can, in an example, form a generally continuous tubular wall 614A defining a lumen 614B therethrough.

FIGS. 7A-7C illustrate an example of insertion of an example of a restrictor apparatus 714, in the form of a moldable stent, into an arteriovenous graft 202. In an example, a deflated balloon 715 and the moldable stent 714 can be endovascularly inserted within an arteriovenous graft 202, for instance, using a delivery catheter or a similar delivery technique. In some examples, the stent 714 can be formed from a moldable material, such as metal. In an example, the stent 714 can be formed from stainless steel. In an example, the stent 714 can include a layer of a substantially impermeable material, such as, but not limited to, Dacron or PTFE. The substantially impermeable material layer, in some examples, can stretch when the stent 714 is expanded, as described below, to define a substantially fluid impermeable wall to perform blood flow restriction, as described herein.

Once deployed at a desired location, for instance, within the arteriovenous graft 202, the balloon 715 can be inflated within the moldable stent 714. In an example, the balloon 715 can include an inflated shape (see, e.g., FIG. 7B) having a first section 715A at a first end and a second section 715B at a second end. The first section 715A, in an example, can be substantially conical and converging from the first end toward the second end. The second section 715B can be substantially conical and converging from the second end toward the first end. In a further example, the inflated shape of the balloon 715 includes a third section 715C between the first section 715A and the second section 715B, the third section 715C being substantially cylindrical.

In an example, inflating the balloon 715 within the moldable stent 714 forces the moldable stent 714 outward and take a shape similar to that of the inflated balloon 715. In effect, a wall 714A of the stent 714 substantially assumes the shape of an outer surface of the balloon 715 to define a lumen 714B, which is essentially a “negative shape” of the balloon 715. In an example, the stent 714 can be expanded into engagement with an interior surface of the arteriovenous graft 202, such as to frictionally engage the stent 714 with the arteriovenous graft 202. In other examples, the stent 714 can include mating or other engagement features, such as for mating or otherwise engaging with the arteriovenous graft 202, such as at corresponding engagement features of the arteriovenous graft 202.

In an example, once the stent 714 is positioned and shaped, the balloon 715 can be deflated and removed from within the moldable stent 714. The moldable stent 714 can maintain its shape similar to that of the inflated balloon 715. In an example, the stent 714 can be shaped to form an entry portion 720 that can include a convergent first lumen 740 that tapers to substantially match an interior diameter of an arterial portion of the arteriovenous graft 202. In an example, the stent 714 can be shaped to form an exit portion 724 that includes a divergent second lumen 744 that tapers to substantially match an interior diameter of a venous portion of the arteriovenous graft 202. In another example, the stent 714 can be shaped to form an intermediate portion 722 between the entry portion 720 and the exit portion 724. In an example, the intermediate portion 722 can include a substantially cylindrical third lumen 742. When shaped, in an example, the stent 714 can form a generally continuous tubular wall 714A defining a lumen 714B therethrough. When inserted within the arteriovenous graft 202, in an example, the stent 714, formed such as described above, can provide a restrictor apparatus to function in a manner similar to examples of restrictor apparatuses described herein.

FIGS. 8A-8D illustrate an example of insertion of an example of a restrictor apparatus 814 into an arteriovenous graft 202. In an example, an outer piece 816 of a blood flow restrictor apparatus 814 is endovascularly inserted into the arteriovenous graft 202. In an example, the outer piece 816 can be formed from a shape memory material that can be compressed and retained within a first retractable sleeve 815 for delivery using a catheter or other delivery technique. When uncompressed or otherwise deployed, the outer piece 816 can include a first diameter and a first length. In an example, the outer piece 816 can form a substantially cylindrical tube in which the first diameter is substantially equal to an interior diameter of the arteriovenous graft 202. In an example, the outer piece 816 can be capable of expanding to include a first diameter that is larger than the interior diameter of the arteriovenous graft 202. This allows frictional engagement of the outer piece 816 with the arteriovenous graft 202.

In an example, an inner piece 818 of the blood flow restrictor apparatus 814 can be endovascularly inserted within the outer piece 816. In an example, the inner piece 818 can be formed from a shape memory material that can be compressed and retained within a second retractable sleeve 817 such as for delivery using a catheter or other delivery technique. When allowed to decompress or otherwise deployed, the inner piece can include a shaped inner profile including a convergent first portion 820 that tapers to substantially match an interior diameter of an arterial portion of the arteriovenous graft 202 and a divergent second portion 824 that tapers to substantially match an interior diameter of a venous portion of the arteriovenous graft 202. In an example, the shaped profile of the inner piece 818 includes a third portion 822 between the first portion 820 and the second portion 824, the third portion 822 including a substantially cylindrical lumen portion.

In an example, a distal end of the inner piece 818 can be attached to a distal end of the outer piece 816. In an example, the distal end of the inner piece 818 can be attached to the distal end of the outer piece 816, such as by attaching an engagement feature of one of inner and outer pieces 818, 816 with a mating engagement feature of the other of the inner and outer pieces 818, 816. In some examples, the engagement features can include, but are not limited to, mating hooks or clasps, magnets, mating detents, pins, docking mechanisms, or adhesive surfaces. In an example, the distal end of the inner piece 818 can be frictionally engaged to the distal end of the outer piece 816.

In an example, a proximal end of the inner piece 818 can be attached to a proximal end of the outer piece 816. In an example, the proximal end of the inner piece 818 can be attached to the proximal end of the outer piece 816 by attaching an engagement feature of one of inner and outer pieces 818, 816 with a mating engagement feature of the other of the inner and outer pieces 818, 816. In some examples, the engagement features can include, but are not limited to, mating hooks or clasps, magnets, mating detents, pins, docking mechanisms, or adhesive surfaces. In an example, the proximal end of the inner piece 818 can be frictionally engaged to the proximal end of the outer piece 816.

In this way, in an example, the outer and inner pieces 816, 818 can be joined to form a substantially unitary structure with the inner piece 818 of the blood flow restrictor apparatus 814 forming a generally continuous tubular wall 814A defining a lumen 814B therethrough. When inserted within the arteriovenous graft 202, in an example, the blood flow restrictor apparatus 814, formed as described above, can function in a manner similar to examples of restrictor apparatuses described herein.

FIGS. 9A-9D illustrate an example of insertion of an example of a restrictor apparatus 914 into an arteriovenous graft 202. In an example, a deflated balloon 915, a shape memory apparatus 916, and a moldable stent 918 or other such moldable apparatus can be endovascularly inserted within an arteriovenous graft 202, for instance, using a delivery catheter or a similar delivery technique. In certain examples, the shape memory apparatus 916 can be stent-like in configuration. In some examples, the shape memory apparatus 916 can be formed from one or more materials including, but not limited to, a shape memory metal, such as nitinol. In some examples, the shape memory apparatus 916 can include a substantially impermeable coating, membrane, or other material, such as, for instance, Dacron or polytetrafluoroethylene (PTFE). The substantially impermeable material, in some examples, can stretch when the shape memory apparatus 916 is expanded, such as described herein, such as to define a substantially fluid impermeable wall to perform blood flow restriction, such as described herein. The shape memory apparatus 916 can include a shape memory metal with a substantially impermeable material coating, sheath, or surface, such as to inhibit or prevent blood or other fluids from passing through a generally tubular wall 914A of the restrictor apparatus 914. In some examples, the moldable stent 918 can be formed from a moldable material, such as metal. In an example, the moldable stent 918 can be formed from stainless steel.

In an example, a compressed restrictor apparatus 914 can be compressed within a retractable sleeve 917, such as for delivery to a desired location. In an example, the compressed restrictor apparatus 914 can be delivered to a location within the implanted arteriovenous graft 202. In an example, the compressed restrictor apparatus 914 can include the moldable stent 918 disposed around the compressed shape memory apparatus 916, with the deflated balloon 915 disposed within each of the moldable stent 918 and the compressed shape memory apparatus 916.

Once at the desired implant location, the restrictor apparatus 914 can be released, such as to allow the shape memory apparatus 916 to uncompress and take a desired shape within the arteriovenous graft 202. In an example, the shape memory apparatus 916 can be released, such as by retracting the retractable sleeve 917 and allowing portions of the shape memory apparatus 916 unconstrained by the moldable stent 918 to assume the desired shape. In an example, ends 916A, 916B of the shape memory apparatus 916, which extend outwardly from the moldable stent 918, are capable of expanding to a maximum diameter in which diameters of ends 916A, 916B of the shape memory apparatus 916 are larger than a diameter of the arteriovenous graft 202 when the shape memory apparatus 916 is unconstrained. This can be used to create a frictional engagement of the outer diameter of the ends 916A, 916B of the shape memory apparatus 916 and the inner diameter of the arteriovenous graft 202 when the shape memory apparatus 916 is released therein.

Once deployed at a desired location, for instance, within the arteriovenous graft 202, the balloon 915 can be inflated within the moldable stent 918. In an example, the balloon 915 can include a substantially cylindrical inflated shape. In an example, inflating the balloon 915 within the moldable stent 918 forces the moldable stent 918 and the portion of the shape memory apparatus 916 to expand and take a substantially cylindrical shape, such as of a desired blood flow restrictive inner diameter.

In an example, once the restrictor apparatus 914 is positioned and shaped, the balloon 915 can be deflated and removed from within the restrictor apparatus 914. In an example, the restrictor apparatus 914 can be shaped to form an entry portion 920 that can include a convergent first lumen 940 that tapers to substantially match an interior diameter of an arterial portion of the arteriovenous graft 202. In an example, the restrictor apparatus 914 can be shaped to form an exit portion 924 that includes a divergent second lumen 944 that tapers to substantially match an interior diameter of a venous portion of the arteriovenous graft 202. In another example, the restrictor apparatus 914 can be shaped to form an intermediate portion 922 between the entry portion 920 and the exit portion 924. In an example, the intermediate portion 922 can include a substantially cylindrical third lumen 942. When shaped, in an example, the restrictor apparatus 914 can form a generally continuous tubular wall 914A defining a lumen 914B therethrough. When inserted within the arteriovenous graft 202, in an example, the restrictor apparatus 914, formed such as described above, can provide a restrictor apparatus to function in a manner similar to examples of restrictor apparatuses described herein.

As particularly described with respect to the examples herein, restrictor apparatuses can be implemented within existing arteriovenous grafts 202 already implanted within a subject. However, in some examples, the restrictor apparatuses described above can be positioned within the arteriovenous graft 202 before implanting the arteriovenous graft 202, such as into a human or animal subject.

FIG. 10 is a summary chart 1000 from a computer simulation comparing the simulated blood flow properties within a subject 104 (FIG. 1) and in an arteriovenous graft 202 or an arteriovenous graft system 200 (FIG. 2B) (including an arteriovenous graft 202 and a restrictor apparatus 214 (FIG. 2B)) implanted within the subject. Line 1002 of the summary chart 1000 lists the simulated blood flow properties occurring within the subject 104 and in the arteriovenous graft 202 (which does not include a restrictor apparatus 214). Lines 1004 and 1006 of the summary chart 1000 list the simulated blood flow properties occurring within the subject 104 and in arteriovenous graft systems 200 including restrictor narrowed portions 322 (see, e.g., FIG. 3B) of 25 millimeters and 45 millimeters in length, respectively. The computer simulation according to this example assumes a graft tubular interior diameter of about 6 millimeters, an effective interior diameter 328 of the restrictor narrowed portion 322 of about 2.5 millimeters, and a divergent exit angle 332 of about 6 degrees with respect to a coaxial central axis of the restrictor apparatus 214.

As shown, the peripheral blood steal 1008 occurring within the subject 104 implanted with a non-restrictive arteriovenous graft 202 is simulated as being much greater than the peripheral blood steal 1008 occurring within the subject 104 implanted with a restrictive arteriovenous graft system 200. More specifically, the peripheral blood steal 1008 occurring within the subject 104 implanted with the arteriovenous graft system 200 including a 25 millimeter long restrictor narrowed portion 322 was simulated as being about 33% less than the peripheral blood steal 1008 occurring within the subject 104 implanted with the non-restrictive arteriovenous graft 202; while the blood steal 1008 within the subject 104 implanted with the arteriovenous graft system 200 including a 45 millimeter long restrictor narrowed portion 322 was simulated as being about 42% less the peripheral blood steal 1008 occurring within the subject 104 implanted with the non-restrictive arteriovenous graft 202.

According to at least one study, such as is found in Sutera, S. P. and Mehrjardi, M. H., Deformation and Fragmentation of Human Red Blood Cells in Turbulent Shear Flow, Biophysical Journal, Vol. 5 (1975): 1-10, wall shear stress 1010 in an arteriovenous graft 202 or graft system 200 should be less than approximately 2000 dynes/centimeter². As shown in the summary chart 1000, the wall shear stress 1010 is 135 dynes/centimeter² and 400 dynes/centimeter² in the non-restrictive arteriovenous graft 202 and the restrictive arteriovenous graft system 200, respectively.

Using information about the wall shear stress 1010, platelet stimulation factor 1012 and predicted percent hemolysis 1014 can be calculated. The platelet stimulation factor 1012 can be calculated by taking the product of (wall shear stress)×(blood residence time in the arteriovenous graft)^{0.452}. According to Wootton, D. M. and Ku, D. N., Fluid Mechanics of Vascular Systems, Diseases, and Thrombosis, Annu. Rev. Biomed. Eng. (1999) 01:299-329, the platelet stimulation factor 1012 should be less than 1000. As shown in the summary chart 1000, the platelet stimulation factor 1012 is 200 and 650 in the non-restrictive arteriovenous graft 202 and the restrictive arteriovenous graft system 200, respectively. The predicted percent hemolysis 1014 can be estimated using a model formula proposed by Giersiepen, M., Wurzinger, L. J., Opitz, R., and Reul, H., Estimation of Shear Stress-Related Blood Damage in Heart Valve Protheses—in vitro Comparison of 24 Aortic Valves, The International Journal of Artificial Organs 13.5 (1990): 300-306. According to Giersiepen et al., the predicted percent hemolysis 1014 is equal to the product of (3.62×10⁻⁵)×(wall shear stress (in Pa))^{2.416}×(blood residence time in the arteriovenous graft)^{0.785}. As shown in the summary chart 1000, predicted percent hemolysis is 2.2, 6.1, and 7.6 in the non-restrictive arteriovenous graft 202, the arteriovenous graft system 200 including the 25 millimeter long restrictor narrowed portion 322, and the arteriovenous graft system 200 including the 45 millimeter long restrictor narrowed portion 322, respectively.

Other simulated information summarized in the chart 1000 includes the maximum strain rate in the arteriovenous graft 1016 and the maximum strain rate at the graft-artery anastomosis 1018. As shown, the maximum strain rate in graft 1016 is simulated as being 2000 and 18000 in the non-restrictive arteriovenous graft 202 and the restrictive arteriovenous graft system 200, respectively; while the maximum strain rate at the graft-artery anastomosis 1018 is simulated as being 20000 and 10000, respectively.

To experimentally illustrate the utility of the present blood flow restrictor apparatus 214, in vivo experiments were performed on three pigs ranging in body weight from about 44.0-47.7 kilograms. In each of the pigs, as respectively shown in FIGS. 11A and 11B, an arteriovenous graft 202 or an arteriovenous graft system 202 (including an arteriovenous graft 202 and a restrictor apparatus 214) was subcutaneously implanted. Each arteriovenous graft 202 extended from an arterial end portion 204 to a venous end portion 208. The arterial end portion 204 was anastomosed 220 to a pig's artery (e.g., iliac artery) 206, while the venous end portion 208 was anastomosed 222 to a pig's vein (e.g., iliac vein) 210.

Each of the pigs was further instrumented with one or more measurement devices, such as one or more blood flow rate detectors 1102A-C, blood pressure detectors, SVO2 detectors, or respiration detectors, for data gathering purposes. Some of the parameters measured by the one or more measurement devices included iliac blood flow upstream to the arteriovenous graft 202, iliac blood flow downstream to the arteriovenous graft 202, blood flow through the arteriovenous graft 202, mean aortic blood pressure, systolic blood pressure, mean iliac venous pressure upstream of the arteriovenous graft 202, continuous cardiac output, continuous cardiac index, and SVO2. FIGS. 11A and 11B illustrate example placement of three blood flow rate detectors 1102A-C used to measure iliac blood flow upstream to the arteriovenous graft 202, iliac blood flow downstream to the arteriovenous graft 202, and blood flow through the arteriovenous graft 202. As shown, a first blood flow rate detector 1102A can be disposed upstream of the arteriovenous graft 202 in the iliac artery 206, a second blood flow rate detector 1102B can be disposed downstream of arteriovenous graft 202 in the iliac artery 206, and a third blood flow rate detector 1102C can be disposed in the arteriovenous graft 202.

Using the three blood flow rate detectors 1102A-C, blood flow rates through each pig were measured with (FIG. 11B) and without (FIG. 11A) the restrictor apparatus 214. In addition, blood flow rates through each pig were measured with and without a dialysis machine 102 present. As discussed above, blood from each pig can be drawn via an arterial cannula 108 (FIG. 2C) at the arterial side 204 of the arteriovenous graft 202 and received by the dialysis machine 102 where it is dialyzed. After being dialysized, the blood can be returned to the pg at the venous side 208 of the arteriovenous graft 202 via a venous cannula 110 (FIG. 2C). For this in vivo experiment, blood was drawn from the arteriovenous graft 202, via the arterial cannula 108, at a rate of 400 milliliters per minute.

FIGS. 11C-11E provide a data chart 1150 summarizing the results of the in vivo experimentation performed on the three pigs. In brief, when the dialysis machine 102 was turned off, it was found that on average blood flow via the arteriovenous graft 202 was reduced (0.51+/−0.03 vs. 0.28+/−0.03 liters/minute) when the restrictor apparatus 214 was present (i.e., integrated with the arteriovenous graft 202 as a unitary body or interposed between the arterial 204 and venous 208 end portions of the arteriovenous graft 202). Without the restrictor apparatus 214 present, the arteriovenous graft 202 on average caused iliac blood flow to increase from 0.15+/−0.12 to 0.61+/−0.12 liters/minute (306.7%). With the restrictor apparatus 214 present, the arteriovenous graft 202 on average caused iliac blood to increase from 0.15+/−0.12 to 0.40+/−0.1 liters/minute (166.7%).

Other information gleaned from the in vivo experimentation performed on the three pigs is as follows. It was found that sufficient blood flow for performing hemodialysis can still be obtained acutely after implanting the restrictor apparatus 214 in the arteriovenous graft 202. Regarding CO (which was measured in two of the three pigs), it was found that the arteriovenous graft 202 caused CO to increase from 3.7 to 4.8 liters/minute (29.7%) and from 2.9 to 3.2 liters/minute (9.4%)—an average increase of 21%—without the restrictor apparatus 214 present. With the restrictor apparatus 214 present, the arteriovenous graft 202 caused CO to increase from 4.1 to 5 liters/minute (22%) and from 2.1 to 2.5 liters/minute (19.1%)—an average increase of also 21%. It was further found that arterial pressure, systolic aortic pressure, and mean iliac venous pressure were not substantially altered depending on whether or not the restrictor apparatus 214 was or was not present.

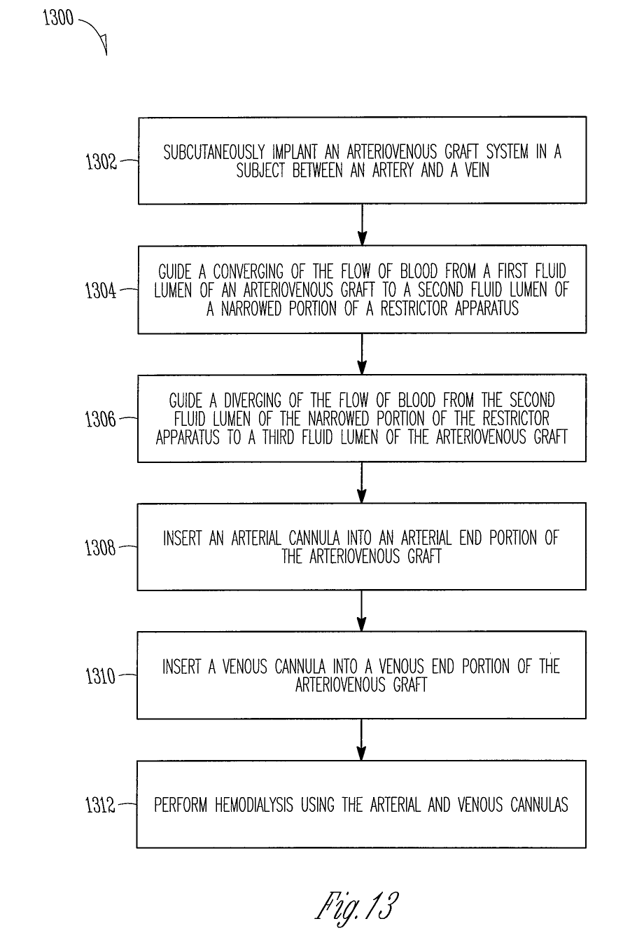

FIG. 12 illustrates an example method 1200 of forming an arteriovenous graft system. At 1202, a restrictor apparatus having fixed dimensions, when implanted, is formed. According to varying examples, forming the restrictor apparatus comprises forming a restrictor entry portion, a restrictor exit portion, and a optionally a restrictor narrowed portion therebetween. The restrictor entry portion includes a convergent first lumen that tapers outward on a first end to substantially match an interior diameter of an arterial portion of an arteriovenous graft. In one example, the first lumen includes an entry angle of less than or equal to about 6 degrees between the wall of the first lumen and a coaxial axis defining a center of the first lumen. In another example, the first lumen includes a convergent curved wall having a radius of curvature of at least 2 millimeters.

Options for the restrictor exit and restrictor narrowed portions are as follows. In varying examples, the restrictor exit portion includes a divergent second lumen that tapers outward on a second end to substantially match an interior diameter of a venous portion of the arteriovenous graft. In one example, the second lumen includes an exit angle of less than or equal to about 6 degrees between the wall of the second lumen and a coaxial axis defining a center of the second lumen. In varying examples, the restrictor narrowed portion includes a third lumen connecting the first and second lumens. The third lumen is narrower than at least a portion of the first and second lumens and substantially matches adjacent interior diameters of the first and second lumens (i.e., substantially matches a second end of the first lumen and a first end of the second lumen). In one example, the third lumen includes an effective interior diameter of at least 2.5 millimeters.

At 1204, the restrictor apparatus is incorporated with an arteriovenous graft. According to certain examples, the incorporation of the restrictor apparatus with the arteriovenous graft includes cutting the arteriovenous graft between an arterial and a venous end portion thereof and securely coupling the restrictor apparatus to such graft portions. According to other examples, the incorporation of the restrictor apparatus with the arteriovenous graft includes disposing the restrictor apparatus within an interior diameter wall of the arteriovenous graft. According to still other examples, the incorporation of the restrictor apparatus with the arteriovenous graft includes the formation of an arteriovenous graft having an integrated restrictor apparatus. Optionally, at 1206, an interior surface of at least one of the first, second, or third lumens of the restrictor apparatus is coated with a biologically active layer.

FIG. 13 illustrates an example method 1300 of restricting a flow of blood through an arteriovenous graft system including an arteriovenous graft and at least one restrictor apparatus. At 1302, the arteriovenous graft system is subcutaneously implanted within a subject between a subject's artery and vein. The at least one restrictor apparatus is located between an arterial end portion and a venous end portion of the arteriovenous graft. The arterial end portion of the arteriovenous graft is anastomosed to the subject's artery, while the venous end portion of the arteriovenous graft is anastomosed to the subject's vein.

At 1304, a converging of a flow of blood from a first fluid lumen defined by a first interior diameter wall of the arteriovenous graft is guided to a second fluid lumen defined by a fixed interior diameter wall of a narrowed portion of the at least one restrictor apparatus. At 1306, a diverging of the flow of blood from the second fluid lumen defined by the fixed interior diameter wall of the narrowed portion of the restrictor apparatus is guided to a third fluid lumen defined by a second interior diameter wall of the arteriovenous graft.

At 1308, an arterial cannula is inserted into the arterial end portion of the arteriovenous graft, and at 1310, a venous cannula is inserted into the venous end portion of the arteriovenous graft. At 1312, hemodialysis is performed on the flow of blood drawn by the arterial cannula and thereafter, the cleansed blood returned to the subject via the venous cannula. During the hemodialysis, the blood flow bypassing the arterial and venous cannulas through the arteriovenous graft is restricted using the restrictor apparatus. Upon completion of the hemodialysis, the arterial and venous cannulas are removed from the respective arterial and venous end portions of the arteriovenous graft.

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the term “subject” is used to include the term “patient.” In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more features thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

What is claimed is:

1. An apparatus comprising:
at least one blood flow restrictor apparatus extending from a first end to a second end, the blood flow restrictor apparatus including:
a restrictor entry portion, including a fixed dimensioned convergent first lumen, when implanted, that tapers via a convex radius of curvature of at least about 2 millimeters to substantially match an interior diameter of an arterial portion of an arteriovenous graft at the first end; and
a restrictor exit portion, including a fixed dimensioned divergent second lumen, when implanted, that tapers to substantially match an interior diameter of a venous portion of the arteriovenous graft at the second end.

2. The apparatus of claim 1, comprising a restrictor narrowed portion disposed between the restrictor entry portion and the restrictor exit portion, the restrictor narrowed portion including a fixed, substantially constant dimensioned third lumen connecting the first and second lumens, the third lumen having a smaller interior diameter than at least a portion of the first and second lumens.

3. The apparatus of claim 2, wherein an axial center of the restrictor narrowed portion is located offset from a midpoint of the first and second ends of the blood flow restrictor apparatus.

4. The apparatus of claim 2, wherein the fixed, substantially constant dimensioned third lumen is at least about 25 millimeters in length.

5. The apparatus of claim 2, wherein the interior diameter of the third lumen is at least about 1.5 millimeters.

6. The apparatus of claim 2, comprising a biologically active layer on an interior surface of at least a portion of at least one of the first lumen of the restrictor entry portion, the second lumen of the restrictor exit portion, or the third lumen of the restrictor narrowed portion.

7. The apparatus of claim 1, comprising:
the arterial portion of the arteriovenous graft, sized and shaped to be coupled to the restrictor entry portion; and
the venous portion of the arteriovenous graft, sized and shaped to be coupled to the restrictor exit portion;
wherein the arterial and venous portions of the arteriovenous graft have a substantially similar internal diameter.

8. The apparatus of claim 7, wherein the restrictor apparatus comprises a structure that is separate from, but couplable to, at least one of the arterial portion of the arteriovenous graft or the venous portion of the arteriovenous graft.

9. The apparatus of claim 8, comprising at least one annular clamp sized and shaped to be disposed around a portion of the arteriovenous graft and a reduced diameter portion of the restrictor apparatus to couple the arteriovenous graft to the at least one restrictor apparatus.

10. The apparatus of claim 1, wherein the outward taper of the divergent second lumen of the restrictor exit portion includes an exit angle, with respect to a coaxial central axis of the second lumen, of less than or equal to about 6 degrees.

11. The apparatus of claim 1, wherein the restrictor apparatus is inserted into the arteriovenous graft in a compressed size and shape and assumes an uncompressed size and shape, including the restrictor entry portion and the restrictor exit portion, when secured in an implanted position.

12. The apparatus of claim 11, wherein the restrictor apparatus is biased outward from the compressed size and shape to the uncompressed size and shape.

13. An apparatus comprising:
at least one blood flow restrictor apparatus extending from a first end to a second end, the blood flow restrictor apparatus including:
a restrictor entry portion, including a fixed dimensioned convergent first lumen, when implanted, that tapers to substantially match an interior diameter of an arteriovenous graft at the first end; and
a restriction exit portion, including a fixed dimension divergent second lumen, when implanted, that tapers at an exit angle, with respect to a coaxial central axis of the second lumen, of less than or equal to about 6 degrees to substantially match the interior diameter of the arteriovenous graft.

14. The apparatus of claim 13, wherein the convergent first lumen of the restrictor entry portion includes an entry angle, with respect to a coaxial central axis of the first lumen, of less than or equal to about 6 degrees.

15. The apparatus of claim 13, comprising a restrictor narrowed portion disposed between the restrictor entry portion and the restrictor exit portion, the restrictor narrowed portion including a fixed, substantially constant dimensioned third lumen connecting the first and second lumens.

16. The apparatus of claim 13, comprising:
an arterial portion of the arteriovenous graft, sized and shaped to be coupled to the restrictor entry portion; and
a venous portion of the arteriovenous graft, sized and shaped to be coupled to the restrictor exit portion.

17. The apparatus of claim 13, wherein the restrictor apparatus is inserted into the arteriovenous graft in a compressed sized and shape and assumes an uncompressed size and shape, including the restrictor entry portion and the restrictor exit portion, when secured in an implanted position.

18. The apparatus of claim 17, wherein the restrictor apparatus is biased outward from the compressed size and shape to the uncompressed size and shape.

19. A method of restricting a flow of blood comprising:
guiding a converging of the flow of blood from a first fluid lumen defined by a first interior diameter wall of an arteriovenous graft to a second fluid lumen defined by a fixed, substantially constant interior diameter wall of a narrowed portion of at least one restrictor apparatus; and
guiding a diverging of the flow of blood from the second fluid lumen defined by the fixed, substantially constant interior diameter wall of the narrowed portion of the restrictor apparatus to a third fluid lumen defined by a second interior diameter wall of the arteriovenous graft;
wherein the narrowed portion includes a fixed interior diameter of at least about 1.5 millimeters and a length of at least about 25 millimeters.

20. The method of claim 19, wherein guiding the converging of the flow of blood includes flowing blood over a convex radius of curvature of at least about 2 millimeters.

21. The method of claim 19, comprising:
inserting an arterial cannula into an arterial end portion of the arteriovenous graft;
inserting a venous cannula into a venous end portion of the arteriovenous graft;
performing hemodialysis using the arterial and venous cannulas;
using the restrictor apparatus located between the arterial and venous cannulas to restrict blood flow bypassing the arterial and venous cannulas through the arteriovenous graft during the hemodialysis; and
removing the arterial and venous cannulas from the respective arterial and venous end portions.

22. The method of claim 21, wherein restricting blood flow includes permitting blood flow through the arterial and venous cannulas of at least about 300 cubic centimeters per minute during the hemodialysis.

23. The method of claim 21, wherein restricting blood flow includes permitting blood flow through the arterial and venous cannulas of at least about 400 cubic centimeters per minute during the hemodialysis.

24. The method of claim 19, comprising:
endovascularly inserting the restrictor apparatus in a compressed shape into the arteriovenous graft; and
releasing the compressed shape to allow the restrictor apparatus to uncompress.

25. The method of claim 24, wherein endovascularly inserting the restrictor apparatus in the compressed shape includes inserting the restrictor apparatus using a catheter.

26. The method of claim 24, comprising:
endovascularly inserting a deflated balloon within the restrictor apparatus;
inflating the balloon, the balloon including an inflated shape having a first section at a first end and a second section at a second end, the first section being substantially conical and converging from the first end toward the second end, the second section being substantially conical and converging from the second end toward the first end, wherein inflating the balloon within the restrictor apparatus forces the restrictor apparatus to take a shape similar to that of the inflated balloon;
deflating the balloon; and
removing the balloon from within the restrictor apparatus, the restrictor apparatus maintaining the shape similar to that of the inflated balloon.

27. The method of claim 19, comprising:
endovascularly inserting an outer piece of the restrictor apparatus into the arteriovenous graft, the outer piece having a first diameter and a first length;
endovascularly inserting an inner piece of the restrictor apparatus within the outer piece, the inner piece having a shaped inner profile including a convergent first portion that tapers to substantially match the first fluid lumen defined by the first interior diameter wall and a divergent second portion that tapers to substantially match the third fluid lumen defined by the second interior diameter wall;
attaching a distal end of the inner piece to a distal end of the outer piece; and
attaching a proximal end of the inner piece to a proximal end of the outer piece.
